(19) United States
(12) Patent Application Publication
(10) Pub. No.: US 2006/0062729 A1
(43) Pub. Date: Mar. 23, 2006

(54) Enhanced ligand binding to neurotensin receptors

(76) Inventor: Robert E. Carraway, Holden, MA (US)

Correspondence Address: FISH & RICHARDSON PC, P.O. BOX 1022, MINNEAPOLIS, MN, 55440-1022, US

(21) Appl. No.: 11/223,711

(22) Filed: Sep. 9, 2005

Related U.S. Application Data
(60) Provisional application No. 60/608,203, filed on Sep. 9, 2004.

(51) Int. Cl. A61K 51/00; A61K 49/14; A61K 31/5415; A61K 31/455

(52) U.S. Cl. 424/1.49; 424/9.34; 424/9.6; 514/355; 514/225.8

(57) ABSTRACT

Provided are combinations and methods for enhancing ligand binding to neurotensin receptor (NTR). Such combinations and methods are useful for applications such as (a) assessing NTR expression, NTR coupling, and NTR function in vitro, (b) assessing NTR expression or NTR function in vivo, (c) imaging tumors; and (d) targeting therapeutics to NTR expressing cells.

### CROSS-REFERENCE TO RELATED APPLICATIONS

This application claims the benefit of the filing date of U.S. Provisional Patent Application Ser. No. 60/608,203, filed on Sep. 9, 2004. The contents of this prior application are hereby incorporated by reference in their entirety.

### GOVERNMENT FUNDING

This invention was made with government support under Department of Defense grant DAMD17-00-1-0528 and National Institute of Health center grant 5P30-DK32520. The government has certain rights in the invention.

### TECHNICAL FIELD

This invention relates to compositions and methods for enhancing ligand binding to Neurotensin receptor (NTR), such compositions and methods are useful in applications such as tumor imaging and therapeutic targeting.

### BACKGROUND

Neurotensin (NT) is a regulatory peptide originally isolated from bovine hypothalamus. NT is expressed in the central nervous system, in intestinal neurons, and in specialized intestinal endocrine cells. NT appears to function both as a neurotransmitter and a hormone. Normally, NT functions as a stimulant of pancreatic and biliary secretions, a stimulant of colonic motility, and an inhibitor of small bowel and gastric motility. NT also stimulates cell growth of prostate cancer cells (Sehgal et al., Proc. Nat'l. Acad. Sci. USA, 91: 4673-4677, 1994), small-cell lung cancer (Sethi et al., Cancer Res., 52: 2737s-2742s, 1992), and cultured human pancreatic cancer cells (Ishizuka et al., Ann. Surg., 217:439-446, 1993).

High affinity neurotensin receptor (NTR) is overexpressed in human tumors, such as Ewing's sarcomas, myelomas, astrocytomas, and tumors of the lung, colon, ovary, pancreas, and prostate. NTR is expressed at much lower levels, if at all, in normal colonic and prostate tissue. See Elek et al., AntiCancer Res. 20, 53-58, 2000; Maoret et al., Biochem. Biophys. Res. Comm., 203:465-471, 1994; Sehgal et al., 1994, supra.; Seethalakshmi, et al., The Prostate 31:183-192, 1997; and Maoret et al., Int. J. Cancer, 80:448-454, 1999.

### SUMMARY

The present invention is based, at least in part, on the discovery that specific subsets of antioxidants, apparently acting through a variety of cellular mechanisms, can enhance the binding of neurotensin (NT) to neurotensin receptor (NTR). Thus, these antioxidants can be used in combination with NTR ligands to (i) enhance the sensitivity of imaging techniques for detecting cells that express NTR (e.g., tumor cells) and (ii) improve the targeting of agents (e.g., toxins) to cells that express NTR (e.g., tumor cells).

For example, in one aspect, the invention features new imaging methods, in which a subject is administered one or more antioxidants or calcium channel blockers (CCBs) and one or more labeled NTR ligands, at least a portion of the subject is subsequently imaged, and the imaged portion is evaluated for a concentration of labeled NTR ligand that exceeds the concentration of labeled NTR ligand in surrounding tissues. High concentrations of NTR ligand indicate the presence of NTR-expressing cells, e.g., tumor cells. The amount of detected NTR ligand can also be used to assess tumor mass. Furthermore, tumors can be repetitively imaged to generate a profile of responsiveness to a set of antioxidants, which can give information about the type, stage, or metabolic activity of the tumor. As another example, in treatment methods described herein, one or more antioxidants or CCBs and one or more NTR ligands conjugated to a tumor-killing agent can be administered to a subject. The co-administered antioxidant or CCB can improve the selective delivery of NTR ligand-conjugated anti-tumor agents to NTR-expressing tumor cells.

In another aspect, the invention features new methods of screening for a cell expressing NTR. These methods include (i) contacting a cell or tissue with a neurotensin receptor ligand (ii) contacting the cell with a CCB or an antioxidant belonging to one of the classes of antioxidants listed herein, and (iii) monitoring binding of neurotensin receptor ligand the cell.

In another aspect, the invention includes new methods for evaluating in vivo NTR function in a target tissue that normally expresses NTRs. These methods include (i) administering to a subject a labeled NTR ligand and a CCB or an antioxidant belonging to one of the classes of antioxidants listed herein. Subsequently, at least a portion of the subject is imaged. The imaged portion includes a target tissue that normally expresses NT receptors at a higher concentration than surrounding tissues. Detection of a target tissue with a higher concentration of label, relative to surrounding non-NTR expressing tissues, indicates that the subject has normal target tissue NTR function. Failure to detect a higher concentration of label in the target tissue, relative to surrounding tissues, indicates the subject does not have normal target tissue NTR function.

In yet another aspect, the invention features new methods for detecting tumors, e.g., Ewing's sarcomas, myelomas, astrocytomas, lung tumors, colon tumors, ovarian tumors, pancreatic tumors, or prostate tumors, that expresses a higher density of NT receptors relative to surrounding tissues in a subject. These methods include administering to the subject a labeled neurotensin receptor ligand and an antioxidant belonging to one of the classes of antioxidants listed below. Subsequently, at least a portion of the subject is imaged. An increased concentration of label within the imaged portion of the subject relative to tissues surrounding the increased concentration, can indicate that the subject has a tumor which expresses a higher density of neurotensin receptors than the surrounding tissues.

In a different aspect, the invention includes methods for treating a tumor by administering to a subject (i) an antioxidant or a CCB and (ii) a neurotensin receptor ligand conjugated to an anti-tumor agent (e.g., a chemotherapeutic, a radiotherapeutic, a proapoptotic agent, a cytotoxic agent, or a cytostatic agent). By enhancing binding of conjugated NTR ligands to cells expressing NTR, the method enhances the specific delivery of anti-tumor agents to NTR-expressing tumor cells.

Antioxidants that can be used in the methods described herein include, but are not limited to, members of the following classes of antioxidants: dihydropyridines (DHPs), polyphenolic antioxidants, flavonoids, retinoids, isoprenoids, glycolytic inhibitors, mitochondrial inhibitors, flavoprotein oxidase inhibitors, iron/zinc chelators, protein kinase-C inhibitors, tyrosine kinase inhibitors, inhibitors of glycogen synthase kinase, and estrogen agonists. Exemplary members of these classes include: felodipine, nicardipine, nitrendipine, nimodipine, nifedipine (NIF), Compound-1, resveratrol, luteolin, apigenin, and quercetin, 2-deoxy-glucose, antimycin-A, rotenone, p-trifluoromethoxyphenyl hydrazone (FCCP), diphenylene-iodonium (DPI), SKF-96365, o-phenanthroline, bis-indoylmaleimide (BIS), rottlerin, Go-6983, genistein, AG1478, MK886, retinoic acid, Rev5901, AA861, nordihydroguaiaretic acid (NDGA), caffeic acid phenethyl ester (CAPE), gossypol, 5,8,11,14-eicosatetraynoic acid (ETYA), estradiol, and diethylstilbesterol (DES). These antioxidants and members of these classes of antioxidants enhance NTR ligand binding to NTR, thereby increasing the sensitivity or selectivity of screens or therapies that employ NTR ligands.

Calcium channel blockers that can be used in the methods described herein include blockers of voltage-gated and store-operated Ca²⁺ channels (VGCC and SOCC). Exemplary CCBs include: felodipine, nicardipine, nitrendipine, nifedipine (NIF), nimodipine, phloretin, verapamil, SKF-96365, miconazole, trifluoperazine, chlorpromazine, and derivatives thereof. These and possibly other CCBs enhance NTR ligand binding to NTR, thereby increasing the sensitivity or selectivity of screens or therapies that employ NTR ligands.

In some embodiments of the methods described herein are screens or therapies for mammalian cancer cells, e.g., Ewing's sarcoma cells, myeloma cells, astrocytoma cells, lung cancer cells, colon cancer cells, ovary cancer cells, pancreas cancer cells, and prostate cancer cells. In some embodiments of the methods described herein, NTR ligands include but may not be limited to: neurotensin; a neurotensin fragment that includes neurotensin (8-13); a neurotensin analog or fragment thereof with a substitution at one or more of the following positions: Arg 8, Arg 9, Pro 10, Tyr 11, Ile 12, or Leu 13, such as MP-2530; a natural relative of neurotensin such as neuromedin N, xenopsin, xenin, and histamine releasing peptide; a neurotensin organomimic such as SR48692, SR142948A, and levocabastine. In further embodiments the NTR ligand is labeled with a reporter group, e.g., a radioactive moiety, a fluorescent moiety, a chromophore, a detectable enzyme, or an antigen.

The invention includes combinations that include one or more neurotensin receptor ligands (e.g., labeled NTR ligand or NTR ligand conjugated with a tumor-killing agent) in combination with a CCB or an antioxidant that is a member of the antioxidant classes disclosed herein. The neurotensin receptor ligands, CCBs, and/or antioxidants can be present in one composition, e.g., mixed in one container. In alternative embodiments, the neurotensin receptor ligands, CCBs, and/or antioxidants are present in separate compositions, e.g., in separate containers. When the receptor ligands, CCBs, and/or antioxidants are present in separate compositions, they may be packaged together.

Also provided herein are kits that include (i) one or more neurotensin receptor ligands (e.g., labeled NTR ligand or NTR ligand conjugated with a tumor-killing agent) and (ii) a CCB or an antioxidant that is a member of the antioxidant classes disclosed herein. In these kits the (i) the NTR ligand and (ii) antioxidant are packaged for administration as a tumor imaging agent or an anti-tumor agent.

As used herein, neurotensin receptor (NTR) refers to neurotensin receptor subtypes, such as NTR1, NTR2, and NTR3, and other receptors whose binding to NTR ligands is subject to antioxidant and/or metabolic feedback mechanisms such that receptor binding or receptor function (e.g., NT-induced formation of inositol phosphates) is altered.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a graph comparing the effect of several dihydropyridines (DHPs) on steady state binding of NT to PC3 cells.

FIG. 1B is a graph showing that Nifedipine (NIF) enhanced specific binding of NT to PC3 cells, but did not affect non-specific binding.

FIG. 1C is a graph showing the relative efficacy of several DHPs for enhancing NT binding.

FIG. 1D is a graph comparing the ability of store-operated (SKF-96365, miconazole) and non-specific (trifluoperazine) calcium channel blockers (CCBs) to enhance NT binding.

FIG. 2A is a graph showing NT-induced inositol phosphate (IP) formation in PC3 cells.

FIG. 2B is a graph showing that DHPs inhibited NT-induced IP formation in PC3 cells.

FIG. 3A is an image of an immunoblot that used the anti-NTR1 antibody described herein to western blot for NTR1 in rat brain extracts.

FIG. 3B is a chart showing that CCBs enhanced cross-linking of radiolabeled NT into NTR1, as assessed by immunoprecipitation.

FIG. 4A is a chart showing that NIF similarly enhanced NT binding to PC3 cells at 4° C., at 37° C., and in the presence of phenylarsine oxide (PASO).

FIG. 4B is a chart showing that NIF enhanced NT binding to NTR1 (i) on the surface of the cell and (ii) internalized NTR1.

FIG. 5A is a chart showing that pretreatment of cells with NIF inhibited NT-mediated IP formation by ˜69%, whereas NIF inhibited bombesin-mediated IP formation by only ˜19%, and ATP-induced IP formation was not affected by NIF.

FIG. 5B is a chart showing the inhibitory effect of 15 μM NIF on NT-, bombesin-, and ATP-induced IP formation in PC3 cells as a percent of IP formation over basal IP levels.

FIG. 6A is a graph showing that NIF inhibited NT-induced calcium influx, whereas NIF did not affect basal calcium influx.

FIG. 6B is a graph showing that (i) NT-induced IP formation depends on the presence of calcium in growth media, (ii) calcium chelators prevent NT-induced IP formation, and (iii) NIF inhibits NT-induced IP formation to the same extent as calcium depletion.

FIG. 7 is a graph showing that calcium chelators and ionophores do not affect NIF-mediated enhancement of NT binding to PC3 cells, showing that NT binding is not dependent on calcium.

FIG. 8A is a graph showing that voltage gated calcium channel (VGCC) agonists ((−) BayK-8644, FPL-64176) and VGCC antagonists (nifedipine, nicardipine, felodipine) both enhance NT binding. Thus, enhanced NT-binding is not a function of calcium movement through these channels.

FIG. 8B is a graph showing that a VGCC agonist ((−) BayK-8644) and a VGCC antagonist (NIF) enhanced NTR1 binding affinity for NT, and did not increase NTR number.

FIG. 8C is a graph showing that VGCC antagonists also enhanced NT-mediated IP formation.

FIG. 8D is a graph showing that increased doses of VGCC antagonist (NIF) or agonist (BayK) shifted the dose response of NT-mediated IP formation downward (indicating that they affect the efficacy of NT-mediated IP formation, not the potency).

FIG. 9A is a graph showing that nimodipene (NIM) and FPL-64176 enhanced NT binding in an additive manner at low concentrations.

FIG. 9B is a graph showing that NT-induced IP formation was inhibited by combined doses of NIM and FPL-64176 in an additive manner at lower concentrations; inhibition of NT-mediated IP formation peaked at 70% inhibition.

FIG. 10A is a graph showing that luteolin, resveratrol, and diphenylene iodonium (DPI) enhanced NT binding to PC3 cells.

FIG. 10B is a graph showing that luteolin and resveratrol enhanced NTR affinity, not NTR number.

FIG. 10C is a graph showing luteolin, resveratrol, and DPI inhibited NT-mediated IP formation in PC3 cells.

FIG. 10D is a graph showing that luteolin, resveratrol, and DPI inhibited NT-mediated IP formation in a dose-dependent decrease in NT-efficacy.

FIG. 11 is a group of chemical structures of different antioxidants.

FIG. 12A is a reaction scheme for hydrogen donation by NADH.

FIG. 12B is a possible reaction scheme for hydrogen donation by DHPs.

FIG. 12C is another possible reaction scheme for hydrogen donation by DHPs.

FIG. 13 is an image of an immunoblot showing that PC3 cells expressed 5-lipoxygenase (5-LOX) and 12-lipoxygenase (12-LOX).

FIG. 14A is a graph showing that nordihydroguaiaretic acid (NDGA) enhanced NT binding to PC3 cells.

FIG. 14B is a graph showing that a number of nonselective lipoxygenase (LOX) inhibitors enhanced NT binding to PC3 cells.

FIG. 14C is a graph showing that other nonselective LOX inhibitors also enhanced NT binding to PC3 cells.

FIG. 15A is a graph showing NT-induced IP formation in the absence (control) or presence of indicated LOX inhibitors.

FIG. 15B is a graph showing that NT-induced IP formation is inhibited in a dose-responsive manner.

FIG. 16A is a graph showing that increasing concentrations of NDGA enhanced the amount labeled NT that bound (i) to cell surface and (ii) to internalized NTR receptors.

FIG. 16B is a graph showing that increasing concentrations of MK886 enhanced the amount labeled NT that bound (i) to cell surface and (ii) to internalized NTR receptors.

FIG. 17A is a graph showing the effect of NDGA on NT displacement as a percentage of maximal binding. The displacement curves indicate that NDGA enhanced NTR affinity, not NTR number.

FIG. 17B is a scatchard plot indicating that NDGA enhanced NTR affinity, not NTR number.

FIG. 18 is a graph that shows NDGA enhancement of NT binding in (i) Locke buffer, (ii) Locke+dithiothreitol (DTT), and (iii) Locke in which NaCl was replaced with sucrose.

FIG. 19A is a graph showing the effects of several protein kinase C (PKC) inhibitors on NT binding to PC3 cells.

FIG. 19B is a graph showing the effects of several PKC inhibitors on NT-mediated IP formation in PC3 cells.

FIG. 20 is a schematic diagram of cellular pathways that (i) alter ROS levels in a cell, (ii) modulate NT-binding to NTR, (iii) modulate NT-mediated IP formation, and (iv) influence cellular proliferation. Some inhibitors of these pathways are also shown.

FIG. 21A is a graph showing that quercetin and resveratrol enhanced NT-binding to NTR at similar concentrations as were required for these compounds to demonstrate mitogen activating protein kinase inhibitory activity.

FIG. 21B is a graph showing that quercetin and resveratrol inhibited NT-induced IP formation at similar concentrations as were required for these compounds to demonstrate mitogen activating protein kinase inhibitory activity.

### DETAILED DESCRIPTION

Members of specific classes of antioxidants and calcium channel blockers (CCBs) can be used to enhance binding of neurotensin receptor (NTR) ligands to cells expressing NTR. In the methods disclosed herein, at least one labeled NTR ligand and at least one antioxidant or CCB are contacted to a cell or administered to a subject to thereby (i) identify cells expressing NTR, e.g., tumor cells, or (ii) target therapeutic compounds to cells expressing NTR, e.g., tumor cells. In methods of identifying a tumor cell, one or more antioxidants and one or more NTR ligands are contacted to a cell and the cell is evaluated for (a) NTR ligand binding to the cell and/or (b) NTR ligand-mediated IP formation in the cell. In methods of screening for tumors in a subject, one or more antioxidants or CCBs and one or more labeled NTR ligands are administered to a subject, at least a portion of the subject is imaged to thereby detect the presence or absence of a localized concentration of labeled NTR-ligand in the imaged portion of the subject. In therapeutic methods, one or more antioxidants or CCBs and one or more NTR ligands conjugated to an anticancer agent are administered to a subject to thereby enhance the targeted delivery and binding of the anticancer agent to tumor cells.

Methods of Detecting Cells that Express High Numbers of NTRs

Antioxidants and CCBs that enhance NTR ligand binding to NTR can be used in conjunction with NTR ligands in methods for detecting cells expressing a relatively high number of neurotensin receptors on their surface relative to surrounding tissue, e.g., normal cells, such as in the brain and colon, and cancer cells, such as prostate cancer cells, small-cell lung cancer cells, and human pancreatic cancer cells. The methods described herein contemplate the administration of one or more NTR ligands before, after, or simultaneously with one or more antioxidants or CCBs.

In one method, a pharmaceutical composition that includes a labeled NTR ligand (e.g., an NTR ligand labeled as described herein) and a pharmaceutical composition that includes an antioxidant or CCB (that enhances NTR ligand binding to NTR) are administered to a subject to detect the presence or absence of cancer cells that express a higher number of NTRs per cell than non-cancerous tissue surrounding the cancer cells. In some embodiments, the labeled NTR ligand is administered before (e.g., up to 1, 2, 4, or 8 hours), concurrent with, or after (e.g., up to 1, 2, 4, or 8 hours) the antioxidant or CCB. In a variation of the method, a subject is administered a pharmaceutical that comprises both (i) a labeled NTR ligand and (ii) an antioxidant or CCB that enhances NTR ligand binding to NTR to detect the presence or absence of NTR-expressing cancer cells. A subject can be a human or an animal, e.g., a human or animal that is (i) in need of screening for cancer, (ii) suspected to have cancer, or (iii) diagnosed with cancer. After administration of one or more pharmaceutical compositions that include antioxidant or CCB and labeled NTR ligand, the subject is imaged using a method appropriate for detecting the labeled NTR ligand, as described in further detail below. The labeled NTR ligand and/or composition including an antioxidant or CCB can be administered by bolus injection or by infusion over a period of time, e.g., greater than two hours. A concentration of labeled NTR ligand indicates that the subject has a number of cells expressing a higher number of NTR, compared to cells in surrounding tissues. Thus, a concentration of labeled NTR ligand in a tissue that does not normally express high levels of NTR can be an indication that a subject has a tumor characterized by cells that overexpress NTR. Similarly, the absence of a concentration of labeled NTR ligand, revealed by imaging, can indicate that a patient does not have a tumor characterized by cells that overexpress NTR.

The expression of NTR in normal tissues (e.g., brain and colon) can also be measured and the results could yield information about normal NTR receptor expressing tissue function.

The magnitude of the response to the antioxidant agent or calcium channel blocker can be used to obtain information about the type, stage, or metabolic state of the tumor, or (in the case of normal tissue), the functions of normal tissue. Thus, in some cases a more aggressive and metabolically active tumor may demonstrate a higher magnitude of response to certain antioxidants or calcium channel blockers. Furthermore, by comparing the response of a tumor to a panel of antioxidants or calcium channel blockers that act by different mechanisms, one can glean information about the type, stage or metabolic state of the tumor. For example, the profile of responses of a tumor can differ for steroid-dependent and steroid-independent tumors or for late-stage and early-stage tumors.

In the methods described herein, one, two, or more different types of antioxidants or CCBs that enhance NTR ligand binding to NTR can be administered to a subject in conjunction with a labeled NTR.

Administration of labeled NTR ligands for imaging is described in U.S. Pat. Nos. 6,194,386; 6,312,661, and 6,630,123. The contents of these patents are incorporated by reference herein.

Imaging devices that can be used in the methods disclosed herein include, but are not limited to, X-ray imaging devices, magnetic resonance imaging devices (e.g., Signa Excite 3T from GE Medical Systems, Waukesha, Wis.), phosphorescent imaging devices, gamma cameras (e.g., T.CAM™ Variable Camera from Toshiba American Medical Systems, Tustin, Calif.), and Near-IR CCD cameras (e.g., Cascade 512B, Photometrics, Tucson, Ariz.). When fluorescent markers are conjugated to NTR ligand, the imaging device can also include a light source capable of producing light, e.g., ultraviolet light, that causes the fluorescent marker to fluoresce.

Methods of Treating Cells That Express NTR

In methods of treating hyperproliferative cells that express NTR, e.g., cancer cells, NTR ligands can be conjugated to an anticancer agent and administered to a patient in combination with an antioxidant or CCB that enhances NTR ligand binding to NTR. By enhancing NTR ligand binding to NTR, the combination improves the efficiency of delivery of the conjugated anticancer agent to cells expressing NTR, relative to the delivery of the anticancer agent to other organs and tissues of the patient. The methods described herein contemplate the administration of one or more NTR ligands before, after or simultaneously with one or more antioxidants or CCBs.

In one method, a subject is administered a pharmaceutical composition that includes an NTR ligand conjugated to an anticancer agent and a pharmaceutical composition that includes an antioxidant or CCB that enhances NTR ligand binding to NTR. In some embodiments, the conjugated NTR ligand is administered before (e.g., up to 1, 2, 4, or 8 hours), concurrent with, or after (e.g., up to 1, 2, 4, or 8 hours) the antioxidant or CCB. In a variation of the method, a subject is administered a pharmaceutical composition that contains a combination of both (i) an NTR ligand conjugated to an anticancer agent and (ii) an antioxidant or CCB that enhances NTR ligand binding to NTR. The conjugated NTR ligand and/or composition including an antioxidant or CCB can be administered by bolus injection or by infusion over a period of time, e.g., greater than two hours. A subject can be a human or an animal, e.g., a human or animal that is in need of treatment for a type of cancer characterized by cancer cells that express NTR. An NTR ligand can be conjugated to one or more anticancer agent, such as a chemotherapeutic, a radiotherapeutic, a proapoptotic agent, a cytotoxic agent, or a cytostatic agent.

Examples of anticancer agents include radiotherapeutic isotopes such as ¹⁰³Pd and ¹²⁵I. Other radioisotopes that can be used as anticancer agents include: ¹⁹⁸Au, ¹⁹⁹Au, ⁹⁰Y, ³²P, ¹⁹²Ir; ²⁴¹Am, ¹⁵⁷Gd (for use in boron-neutron capture therapy), ²⁵²Ca, ¹⁸⁸Rh, ¹⁵³Sm, ¹¹¹In, 169Yb, and ¹⁶⁶Ho.

Chemotherapeutics that can be used as anticancer agents include, but are not limited to: adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol™, Bristol-Myers Squibb Oncology, Princeton, N.J.) and doxetaxel (Taxotere™, Rhne-Poulenc Roher, Antony, France), toxotere, methotrexate, cisplatin, melphalan, vinblastine, bleomycin, etoposide, ifosfamide, mitomycin C, mitoxantrone, vincristine (Leurocristine), vinorelbine, carboplatin, teniposide, daunomycin, carminomycin, aminopterin, dactinomycin, mitomycins, esperamicins (see U.S. Pat. No. 4,675,187), melphalan and other related nitrogen mustards. See also Perry, M. C., ed., The Chemotherapy Sourcebook, 3^{rd} Ed., Lippincott Williams & Wilkins (Baltimore, Md., 2001). Chemotherapeutics also include hormonal agents that regulate or inhibit hormonal action on tumors such as tamoxifen and onapristone. In some cases, chemotherapeutics can include cytotoxic substances such as enzymatically active toxins (of bacterial, fungal, plant or animal origin), or fragments thereof.

Administration of NTR ligands conjugated to therapeutic agents is described in U.S. Pat. Nos. 6,194,386; 6,312,661, and 6,630,123. The contents of these patents are incorporated by reference herein.

Neurotensin Receptor Ligands: NT, NT Derivatives, and Other NTR Ligands

In the combinations and methods disclosed herein, at least one antioxidant or calcium channel blocker is combined, or used in conjunction, with at least one NTR ligand. An exemplary NTR ligand is neurotensin, a thirteen amino acid peptide with the following amino acid sequence: pGlu-Leu-Tyr-Glu-Asn-Lys-Pro-Arg-Arg-Pro-Tyr-Ile-Leu (SEQ ID NO:1). Neurotensin, sometimes referred to as NT (1-13), has a Kd of about 0.3 nM for binding to membranes containing the neurotensin-1 receptor (NTR1) and about 2-6 nM at the neurotensin-2 receptor (NTR2) (Zsurger, et al., Brain Res., 586:303, 1992). The C-terminal six residues of NT are most essential for binding to receptors and for biologic activity (Carraway et al. (1975) in Peptides: Chemistry, Structure and Biology, (R. Walter and J. Meienhofer, eds.), Ann Arbor Science Publ., Inc., Ann Arbor, Mich., p. 679-685). Therefore, analogs of NT containing these six residues can be ligands for NTR. Amino acids with similar properties can be substituted at some of the six C-terminal positions (e.g., Lys for Arg, Trp for Tyr, Leu for Ile, Val for Leu), yielding NT derivatives with some ability to bind NTR. All such analogs are potential ligands for the assays described herein. An example of a selective NTR-1 agonist is Trp 11 NT(1-13), which shows a binding affinity of about 1 nM for binding to the neurotensin-1 receptor and about 27 nM for binding to the neurotensin-2 receptor.

NTR ligands include NT derivatives and organomimics that bind to NTR. NT derivatives include NT peptides modified as described herein. NT derivatives include peptides containing a reversed N-terminal to C-terminal amino acid sequence of NT. NT peptide can be modified by substituting one or more amino acids in the peptide with an artificial amino acid analog. For example, NT derivatives can include one or more D-amino acids substituted for the corresponding one or more L-amino acids of NT. In other NT derivatives, substituted artificial amino acid analogs include β-amino acids, β-substituted β-amino acids (“β³-amino acids”), phosphorous analogs of amino acids, such as α-amino phosphonic acids and α-amino phosphinic acids, and amino acids having non-peptide linkages. Artificial amino acids can be used to create peptidomimetics, such as peptoid oligomers (e.g., peptoid amide or ester analogs), β-peptides, cyclic peptides, oligourea or oligocarbamate peptides, or heterocyclic ring molecules.

As the positions Arg 8-Arg 9 and Tyr 11-Ile 12 in NT are known to be susceptible to endopeptidases, these peptide bonds can be altered to stabilize an NTR ligand. For example, a number of NT derivatives in which these peptide bonds have been systematically reduced are described in Couder et al., Int. J. Pept. Protein Res., 41:181 (1993). One NTR derivative is Trp 11 NT(1-13), a peptide that is identical to NT (1-13) except for the substitution of tryptophan for tyrosine at amino acid 11. Another NT derivative, called MP-2530, contains the following substitutions: Ile 12 to t-buGly, Arg 8 to (PipAm)Gly, and Lys 6 to (Pip)Gly. MP-2530 demonstrated increased serum and urine stability while retaining NTR binding affinity (Srinivasan et al., J. Pept. Sci., 6:S184, 2000). Other stabilized NT analogs have been described in Eglio et al., J. Nucl. Med., 40:1913-1917, 1999.

NTR ligands also include NT derivatives that are fragments of NT or extended forms of NT that retain the ability to bind NTR. One example of an NT fragment that binds to NTR is neurotensin (8-13), with the sequence Arg-Arg-Pro-Tyr-Ile-Leu (SEQ ID NO:2), described by Granier et al., Eur. J. Biochem., 124:117-124, 1982. NTR ligands also include NT fragment derivatives, such as the stabilized neurotensin (8-13) pseudopeptide described by Garcia-Garayoa et al., J. Nucl. Med., 43:374-383 (2002). Still other NT fragment derivatives that bind NTR are described in U.S. Pat. No. 6,194,386. NTR ligands include naturally occurring NT-like peptides such as neuromedin-N (Lys-Ile-Pro-Tyr-Ile-Leu; SEQ ID NO:3), xenopsin (pGlu-Gly Lys-Arg-Pro-Trp-Ile-Leu; SEQ ID NO:4), xenopsin-related peptide (Phe-His Pro Lys-Arg Pro-Trp-Ile-Leu; SEQ ID NO:5), histamine releasing peptide (Ile-Ala-Arg-Arg-His Pro-Tyr-Phe-Leu; SEQ ID NO:6), and derivatives thereof.

NT ligands also include organomimics that exhibit specific binding to NT receptors. Organomimics can be produced by and selected from combinatorial chemistry libraries using NTR binding assays. Such compounds are useful in the assays described herein. Some NT ligands that are organomimics have been described: SR48692, a selective antagonist for the neurotensin-1 receptor, has the chemical structure: {2-[1-(7-chloro-4-quinolinyl)-5-(2,6-dimethoxyphenyl) pyrazol-3-yl) carboxylamino] tricyclo (3.3.1.1.^{3.7}) decan-2-carboxylic acid} (Gully, et al., Proc. Natl. Acad. Sci. USA, 90:65, 1993). SR142948A, a non-selective antagonist that binds to both neurotensin-1 and neurotensin-2 receptors, has the chemical structure: 2-(5,6-dimethylaminopropyl)-1-[4-{N-(3-dimethylaminopropyl)-N-methylcarba-moyl}-2-isopropylphenyl]-1H-pyrazole-3-carbonyl)aminoadamantane-2-carboxylic acid (Gully, et al., J. Pharmacol. Exper. Therap., 280:802, 1997). It is conceivable that other organomimics will be found that bind to NTR. Levocabastine is another organic compound that has no obvious structural similarity to NT but is able to bind to NTR, specifically to NTR2. U.S. Pat. Nos. 5,250,558 and 5,204,354 disclose Neurotensin receptor antagonists. U.S. Pat. No. 5,407,916 discloses peptidic Neurotensin agonists.

Labeling of NTR Ligands

NTR ligands useful in the imaging and targeting methods disclosed herein can be labeled with a reporter moiety that allows detection of the NTR ligands in a subject. NTR ligands can be labeled by any of a variety of methods.

In some embodiments, NTR ligands are labeled with contrast agents. Contrast agents are useful to enable or enhance the imaging of labeled NTR ligand using imaging methods such as X-rays, computerized tomography, magnetic resonance imaging (MRI), nuclear imaging, ultrasound, phosphorescence, or fluorescence imaging. For example, NTR ligands can be conjugated to any of a number of existing or novel paramagnetic nanoparticle contrast agents. Conjugation of MRI contrast agents, e.g., gadolinium, has been described, e.g., Flacke et al., Circulation, 104:1280-1285 (2001) and Allen and Meade, J. Biol. Inorg. Chem., 8:746-750 (2003).

In some embodiments, NTR ligands are radiolabeled. NTR1 ligands can be conjugated to radioisotopes such as ^{99m}Tc, 111In, and ⁶⁷Ga, as well as other radioisotope suitable for nuclear imaging, e.g., ¹⁸⁶Re, ¹⁸⁸Re, ⁷¹As, ⁹⁰Y, ⁶⁷Cu, ¹⁶⁹Er, ¹²¹S ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, ¹¹⁴Ag, ¹²⁴I, and ¹³¹I. Radiolabels can be conjugated to a chelator, which is typically placed at the N-terminus of the NTR ligand. Examples of chelators are: acetylmercaptoacetyl-triglycine (MAG₃), diethylenetriaminopentaacetate (DTPA), 1,4,7,10-tetraazacyclododecane-N,N′N″,N′″-tetracetic acid (DOTA), derivatives of DOTA and DTPA, as well as other chelators. See Meyer et al., Invest. Radiol., 25: S53 (1990) and U.S. Pat. Nos. 5,155,215; 5,087,440; 5,219,553; 5,188,816; 4,885,363; 5,358,704; and 5,262,532. In some methods, NTR ligands can be synthesized using one or more radiolabeled amino acids or amino acid analogs.

In other embodiments, NTR ligands are labeled with a chromophore or a fluorescent label. For example, amine-reactive conjugates, e.g., N-hydroxysuccinimide esters or isothiocyanates, of fluorescent labels can be reacted with protein amino groups of an NTR ligand. Maleimide groups of dyes can be reacted with protein sulfhydryl groups on an NTR ligand. Fluorescent labels include near-infrared fluorophores such as Cy5™, Cy5.3™, Cy5.5™, and Cy7™ (Amersham Piscataway, N.J.), Alexa Fluor® 680, Alexa Fluor® 700, and Alexa Fluor® 750, (Molecular Probes Eugene, Oreg.), Licor NIR™, IRDye38™, IRDye78™, and IRDye80™, (LiCor Lincoln, Nebr.), or LaJolla Blue™, (Diatron, Miami, Fla.), and indocyanine green, and the fluorochromes disclosed in U.S. Pat. No. 6,083,875.

In other embodiments, NTR ligands are labeled with contrast agents such as magnetic particles, e.g., superparamagnetic, ferromagnetic, or paramagnetic particles. Paramagnetic metals (e.g., transition metals such as manganese, iron, chromium, and metals of the lanthanide group such as gadolinium), which alter the proton spin relaxation property of the medium around them, can also be used as contrast agents for NTR ligands.

Specific examples of magnetic nanoparticles include monocrystalline iron oxide nanoparticles (MIONs) as described, e.g., in U.S. Pat. No. 5,492,814; Whitehead, U.S. Pat. No. 4,554,088; Molday, U.S. Pat. No. 4,452,773; Graman, U.S. Pat. No. 4,827,945; and Toselson et al., Bioconj. Chemistry, 10:186-191 (1999). These particles can also be superparamagnetic iron oxide particles (SPIOs), ultra small superparamagnetic iron oxide particles (USPIOs), and cross-linked iron oxide (CLIO) particles (see, e.g., U.S. Pat. No. 5,262,176).

In some embodiments, a paramagnetic label is a metal chelate. Suitable chelating moieties include macrocyclic chelators such as 1,4,7,10-tetrazazcyclo-dodecane-N,N′,N″,N′″-tetraacetic acid (DOTA). Gadolinium (Gd³⁺), dysprosium (Dy³⁺), and europium are suitable for use in human patients. Manganese can also be used for imaging tissues other than the brain. In other embodiments, CEST (Chemical Exchange Saturation Transfer) can be used. The CEST method uses endogenous compounds such as primary amines as reporter groups that can be linked to the NTR ligand.

In some of the methods discussed herein, NTR ligands can be labeled with enzymes having a detectable enzymatic activity, such as horseradish peroxidase, alkaline phosphatase, β-galactosidase, or glucose oxidase. In these methods, NTR ligands can be labeled with non-enzymatic proteins that interact with other detectable moieties, e.g., antigens detectable using by labeled antibodies. Examples of detectable antigens include avidin or biotin.

Antioxidants

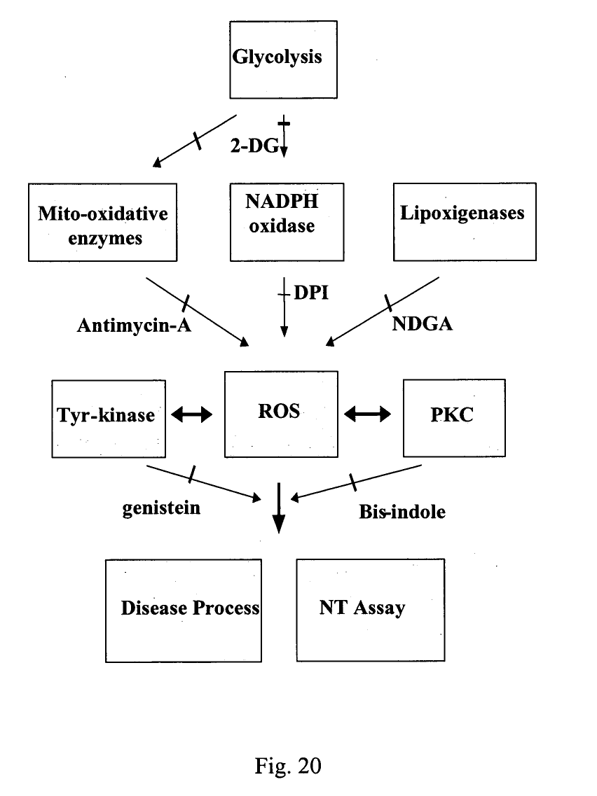

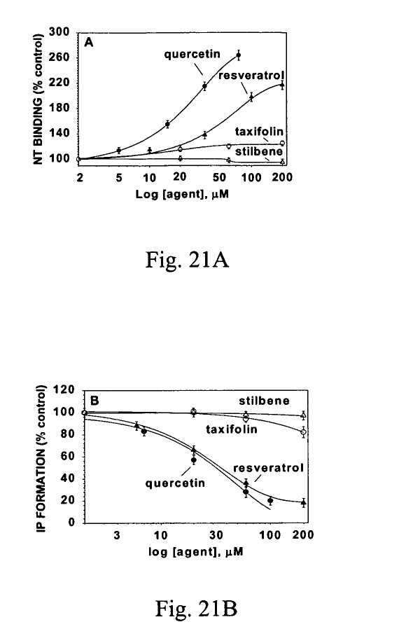

Antioxidants that can be used in the methods and compositions disclosed herein share an ability to accumulate in a cell and reduce the levels of reactive oxygen species (ROS) within the cell. These antioxidants do not necessarily achieve the reduction of ROS by the same mechanisms. For example, antioxidants that can be used in the methods and combinations disclosed herein include antagonists of any one of the ROS producing pathways depicted in FIG. 20.

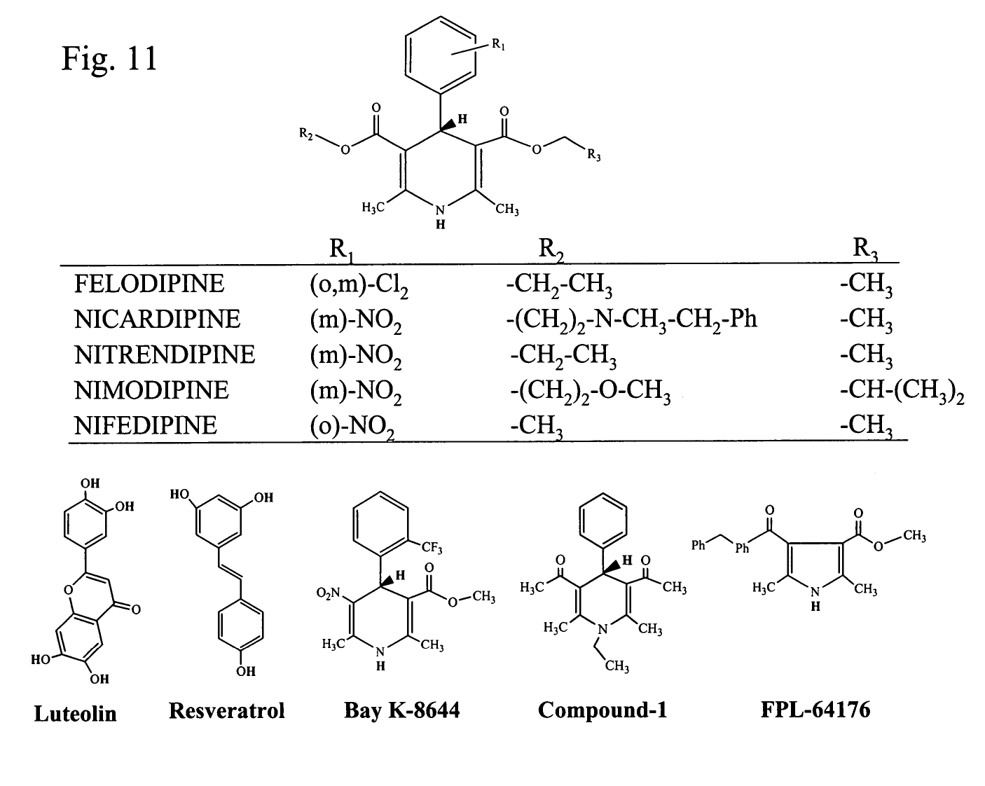

One class of exemplary antioxidants that can be used in the methods and compositions disclosed herein includes members of the class of ROS scavenging 1,4-dihydropyridines (DHPs), e.g., felodipine, nicardipine, nitrendipine, nimodipine, nifedipine, and Compound-1 (an N-ethyl DHP described in Ortiz et al., Pharm. Res., 20:292-296, 2003). Another class of antioxidants includes members of the polyphenolic antioxidants, such as flavonoids and flavonols, e.g., resveratrol, luteolin, apigenin, and quercetin. Members of these two classes of antioxidants enhance NTR ligand binding to NTR, and they share certain structural similarities, namely an aromatic ring structure linked to one or more reactive NH or OH group with redox capacity. See FIG. 11.

Members of a second class of antioxidants that can be used in the methods and combinations disclosed herein are lipoxygenase (LOX) inhibitors, e.g., nor-dihydroguaretic acid (NDGA), MK886, retinoic acid, gossypol, Rev5901, AA861, ETYA, CAPE, LY-171883, CGS-21680, and baicalein.

Other antioxidants that can be used in the methods and compositions disclosed herein are members of any of the following classes of antioxidants: (i) glycolytic inhibitors, e.g., 2-deoxy-glucose; (ii) mitochondrial inhibitors, e.g., antimycin-A, myxathiazole, and p-trifluoromethoxyphenyl hydrazone (FCCP); (iii) flavoprotein oxidase inhibitors, e.g., diphenylene-iodonium (DPI); (iv) iron/zinc chelators, e.g., o-phenanthroline; (v) protein kinase-c inhibitors, e.g., bis-indoylmaleimide, rottlerin, and Go-6983; (vi) tyrosine kinase inhibitors, e.g., genistein, AG1478, and (vii) estrogen agonists, e.g., estradiol and diethylstilbesterol (DES).

Other antioxidants that can be used in the methods and combinations disclosed herein are BayK-8644 and butylated hydroxy anisole (BHA).

Still other antioxidants that can be used in the methods and combinations disclosed herein include antioxidants identified by the methods disclosed for identifying antioxidants that enhance NTR ligand binding to NTR.

Calcium Channel Blockers

Calcium channel blockers (CCBs) that can be used in the methods and combinations disclosed herein include blockers of both voltage-gated and store-operated calcium channels (VGCC and SOCC). Blockers of VGCC include 1,4-dihydropyridines (e.g., nifedipine), phenylalkylamines (e.g., verapamil), and benzothiazepines (e.g., diltiazem). Inhibitors of SOCC include imidazoles (e.g., SKF-96365) and tricyclics (e.g., trifluoperazine). For the effects on NTR binding and function, the preferred CCBs are the 1,4-dihydropyridines and the imidazoles. These agents also exhibit some degree of nonspecificity (Harper et al., Biochem. Pharmacol., 65:329-38, 2003; Triggle, Mini Rev. Med. Chem., 3:217-25, 2003).

Calcium channel blockers that can be used in the methods and combinations described herein include felodipine, nicardipine, nitrendipine, nifedipine (NIF), nimodipine, phloretin, verapamil, flunarizine tetrandrine, SKF-96365, miconazole, and derivatives thereof.

Methods of Identifying Antioxidants that Enhance NTR Ligand Binding to NTR

Methods for identifying antioxidants that enhance NTR ligand binding to NTR are provided herein. In one method, a cell displaying NTR can be contacted with a candidate antioxidant and an NTR ligand, e.g., NT or an NT derivative. The binding characteristics of a specific NTR ligand to the cell is subsequently evaluated, e.g., the specific binding of the NTR ligand (displaceable by NT) is measured. In another example, a cellular membrane containing NTR or a soluble form of NTR is contacted with a candidate antioxidant and an NTR ligand, and the binding characteristics of the NTR ligand to the NTR are measured. Although intact cells appear to be required for the effects described here, it is conceivable that methods can be developed that permit these effects to be measured using cell membranes or a soluble form of NTR. Candidate antioxidants that can be used in these methods include, but are not limited to, antioxidants that have an aromatic ring structure linked to a reactive NH or OH group that has redox capacity. Other candidate antioxidants that can be used in these methods include lipoxigenase inhibitors, glycolytic inhibitors, mitochondrial inhibitors, flavoprotein oxidase inhibitors, iron/zinc chelators, protein kinase-c inhibitors, and tyrosine kinase inhibitors. In these methods, candidate antioxidants that enhance the specific binding of an NTR ligand to the cell or cell membrane (relative to NTR ligand binding in the absence of the antioxidant) are antioxidants that enhance NTR ligand binding to NTR.

Pharmaceutical Compositions

An NTR ligand described herein, e.g., a labeled and/or conjugated NTR ligand, and an antioxidant or CCB (that enhances NTR ligand binding to NTR) can each be incorporated into a separate pharmaceutical composition. Alternatively one or more NTR ligands described herein and one or more antioxidants or CCBs can be combined, e.g., by mixing together, in a single pharmaceutical composition. Pharmaceutical compositions typically include an active compound (i.e., one or more NTR ligands described herein, one or more antioxidants or CCBs, or a combination thereof) and a pharmaceutically acceptable carrier. A “pharmaceutically acceptable carrier” can include solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor™ EL (BASF, Parsippany, N.J.) or phosphate buffered saline (PBS). In all cases, the composition should be sterile and fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride, in the composition. Prolonged absorption of the injectable compositions can be achieved by including an agent which delays absorption, e.g., aluminum monostearate or gelatin, in the composition.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; and a flavoring agent such as peppermint, methyl salicylate, or orange flavoring.

For administration by inhalation, the active compound(s) are delivered in the form of an aerosol spray from a pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams, as generally known in the art.

The active compound(s) can also be prepared in the form of suppositories (e.g., with conventional suppository bases such as cocoa butter and other glycerides) or retention enemas for rectal delivery.

In one embodiment, the active compound(s) are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The skilled artisan will appreciate that certain factors influence the dosage and timing required to effectively treat a patient, including but not limited to the type of patient to be treated, the severity of the disease or disorder, previous treatments, the general health and/or age of the patient, and other diseases present. Moreover, treatment of a patient with a therapeutically effective amount of an active compound can include a single treatment (e.g., for imaging) or, preferably, can include a series of treatments. Appropriate doses of the compound depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a container, pack, or dispenser together with instructions for administration. For example, pharmaceutical composition that includes one or more NTR ligand (labeled or conjugated) can be packaged together with a pharmaceutical composition that includes an antioxidant. Such packaging facilitates the practice of combination therapies disclosed herein.

The invention is further described in the following examples, which do not limit the scope of the invention described in the claims.

### EXAMPLES

### Example 1

### Iodination of Ligands and binding to PC3 Cells

[¹²⁵I]-sodium iodide (2000 Ci/mmol), was obtained from Dupont New England Nuclear (Boston, Mass.). [4-azido-Phe⁶]-NT was synthesized using reagents from Novabiochem (San Diego, Calif.). Chemicals whose origin is unspecified were obtained from Sigma (St. Louis, Mo.).

Ligands were iodinated using the following concentrations: 3 nmol for human epidermal growth factor (EGF) obtained from Calbiochem, San Diego, Calif.; 15 nmol each for neurotensin (NT), [Tyr⁴, Nle¹⁴]-bombesin, pindolol, and des-Gly-[Phaa1, D-Tyr(Et)2, Lys6, Arg8]-vasopressin (HOLVA) obtained from Peninsula, Belmont, Calif. Iodinations were performed using chloramine T (10 μg) as described (Carraway et al., Peptides, 14:37-45, 1993). All reactions were stopped using sodium metabisulfite (30 μg), except for EGF (stopped by dilution). The mono-iodinated products were purified by reverse-phase HPLC using μBondapak™ C18 (3.9×300 mm column from Millipore, Bedford, USA) eluted at 1.5 ml/minutes with a linear gradient (60 minutes) from 0.1% trifluoroacetic acid (TFA) to 60% CH₃CN, 0.1% TFA. The specific activity of the purified 125I-NT was 1000-2000 cpm/fmol as determined by radioimmunoassay (Carraway et al., J. Biol. Chem., 251:7035-7044, 1976).

PC3 cells were obtained from American Type Culture Collection (Manassas, Va.) and maintained as described (Seethalakshmi et al., The Prostate, 31:183-192, 1997). Cells, passaged no more than 30 times, were grown to 95% confluency in 24-well culture plates. For binding studies, cells were washed with 2 ml/well of Hepes-buffered Locke-BSA (Locke): 148 mM NaCl, 5.6 mM KCl, 6.3 mM Hepes, 2.4 mM NaHCO3, 1.0 mM CaCl₂, 0.8 mM MgCl₂, 5.6 mM glucose, and 0.1% bovine serum albumin (BSA); pH 7.4. Equilibrium binding at 37° C. was performed for 25 minutes using 10⁵ cpm/ml of each ¹²⁵I-labeled ligand in 1.0 ml Locke with varying amounts of unlabeled ligand (0-1 μM). The reaction was stopped on ice for 15 minutes, the medium was aspirated and the cells were washed twice with 2 ml and once with 4 ml ice-cold saline. During this 5 minute washing procedure, dissociation of ¹²⁵I-NT from cell receptors was<1%. Total cellular binding was assessed by measuring radioactivity and protein (Bio-Rad assay; BSA standard) in cells extracted in 0.6 ml 0.2 M NaOH. A Packard 10-well γ-counter was used to measure radioactivity. Specific binding was defined as that displaceable by 1 mM ligand. Binding displacement curves were constructed for each set of treatments and binding parameters were determined by Scatchard analysis. Ki was determined by using the equation {Ki=IC50/1+[L]/Kd} where Kd and [L] are the dissociation constant and the concentration of the ligand, respectively (Cheng and Prusoff, Biochem. Pharmacol., 22:3099-3108, 1973).

Specific binding of ¹²⁵I-NT (10⁵ cpm/ml) to PC3 cells at 37° C. was>95% of total binding and was 16.8±0.81 cpm ¹²⁵I-NT bound/mg protein (n=12), which corresponded to ˜3000 cpm ¹²⁵I-NT bound/well. Table 1 gives the binding parameters determined for NT binding to PC3 cells. The data show that labeled ligand, e.g., ¹²⁵I-NT, remained intact during incubation and that dissociation did not occur during washing.

TABLE 1
Parameters Determined for Binding of
¹²⁵I-labeled Ligands to PC3 Cells

| | Specific Binding ^{b} | Bmax ^{c} | Ki ^{c} |
|---|---|---|---|
| Ligand ^{a} | (% of total) | (fmol/mg) | (nM) |
| ¹²⁵I-NT | 95 | 158 ± 9 | 1.0 ± 0.07 |
| ¹²⁵I-[Nle¹⁴]- | 95 | 1016 ± 64 | 0.6 ± 0.09 |
| bombesin | | | |
| ¹²⁵I-EGF | 95 | 151 ± 11 | 0.6 ± 0.07 |
| ¹²⁵I-pindolol | 66 | 86 ± 6 | 0.3 ± 0.05 |
| ¹²⁵I-HOLVA | 77 | 156 ± 12 | 0.5 ± 0.07 |

^{a} NT, [Nle¹⁴]-bombesin and EGF are agonists for NTR1, bombesin receptor and EGF receptor, respectively. Pindolol and HOLVA are antagonists for β2-adrenergic receptor and vasopressin (V1a) receptor, respectively.
^{b} All ligands were HPLC purified (specific activity, >1000 Ci/mmol). Specific binding was measured to near confluent cells (≅185 μg protein/well) using 10⁵ cpm ¹²⁵I-ligand in 1.0 ml Locke (see Methods).
^{c} Scatchard analysis was performed using 12 ligand concentrations and results were from three to nine experiments.

### Example 2

### Calcium Channel Blockers (CCBs) Enhance NT Binding to PC3 Cells

Phloretin was obtained from Calbiochem (San Diego, Calif.). Nimodipine, verapamil, diltiazem, NT, nifedipine (NIF), miconazole, tetraethylammonium (TEA), flunarizine, phenylarsine oxide, amiloride, pindolol were from Sigma (St. Louis, Mo.). Stocks of test agents were prepared daily (10 mM in DMSO) and diluted into Locke just before use, except for SKF-96365, miconazole and trifluoperazine, which were dissolved in Locke. Binding experiments were performed as described in Example 1.

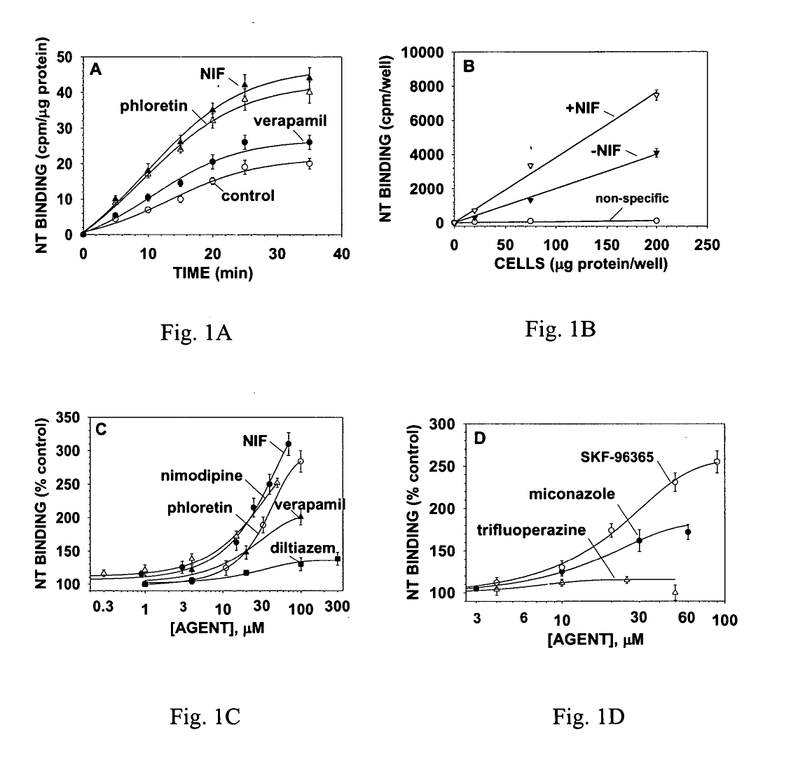

Calcium channel Blockers (CCBs) include blockers of voltage gated calcium channel (VGCC) and store-operated calcium channels (SOCC). VGCC blockers NIF, phloretin, and verapamil were found to increase the apparent rate of and the steady state level of NT binding to PC3 cells (FIG. 1A). NIF enhanced specific binding, without altering non-specific binding and was effective across a 10-fold range in cell density (FIG. 1B). The order of efficacy was NIF>phloretin>verapamil>diltiazem. NT binding was increased as much as 3.1-fold by NIF, 2.9-fold by phloretin, 2.0-fold by verapamil and 1.4-fold by diltiazem (FIG. 1C). A sub-class of VGCC blockers referred to as 1,4-dihydropyridines (DHPs), represented here by nifedipine (NIF) and nimodipine (NIM), were the most potent agents, elevating NT binding at sub-micromolar concentrations {control, 100±4%; 0.3 mM NIM, 116±5% (p<0.05); 0.9 mM NIF, 115±5% (p<0.05)}. Whereas less specific CCBs (flunarizine, tetrandrine, trifluoperazine and chlorpromazine) had only modest effects (Table 2), well-defined blockers of SOCC (SKF-96365, miconazole) enhanced NT-binding up to 2.9-fold (FIG. 1D; Table 2). The data showed that certain CCBs, including DHPs and some SOCC blockers, enhanced NT-binding.

TABLE 2
Activity of CCBs on NT Binding and NT-induced IP Formation

| | | NT Binding ^{a} | | IP Formation ^{b} | |
|---|---|---|---|---|---|
| | | Efficacy | EC50 | Efficacy | IC50 |
| Channel | Agent | (% increase) | (μM) | (% decrease) | (μM) |
| VGCC | NIF ^{c} | 210 | 15 | 74 | 15 |
| | Phloretin ^{c} | 186 | 27 | 70 | 23 |
| | Verapamil ^{c} | 85 | 43 | 58 | 53 |
| | Diltiazem ^{c} | 38 | >300 | nd | nd |
| | flunarizine ^{d} | 45 | >100 | nd | nd |
| | tetrandrine ^{d} | 35 | >100 | nd | nd |
| SOCC | SKF-96365 | 155 | 23 | 69 | 26 |
| | miconazole | 75 | 60 | 54 | 51 |
| | trifluoperazine | 16 | >100 | 14 | >100 |
| | chlorpromazine | 36 | >100 | nd | nd |

^{a} Efficacy was defined as the maximal % increase in NT binding observed for each agent. ED50 was defined as the [agent] at which NT binding was increased by 80%. The data are means determined in 3-8 experiments for each agent.
^{b} Efficacy was defined as the maximal % decrease in NT-induced IP formation observed for each agent. IC50 was defined as the [agent] at which IP formation was decreased by 50%. The data are means determined in 3 to 8 experiments for each agent. nd, not determined.
^{c} L-type CCBs.
^{d} L-type/T-type blockers.

### Example 3

### CCBs Inhibited NT-induced IP Formation in PC3 Cells

[1,2-³H(N)]-myo-inositol (60 mCi/mmol) was obtained from Dupont New England Nuclear (Boston, Mass.). IP formation was measured using [³H]-inositol to label the phosphoinositide pool as described in Chen and Chen, Endocrinology, 140:1639-48, 1999. PC3 cells in 24-well plates were incubated 48 hours with myo-[³H]-inositol (2.5 mCi/ml) in medium 199, 5% fetal calf serum. Medium 199 (Difco, Franklin Lakes, N.J.) was chosen because of its low inositol content. After washing with 2 ml Locke, cells were preincubated 10 minutes with test agents in Locke, 15 mM LiCl, and reactions were started by adding a maximal dose of NT (30 nM) or vehicle. After 30 minutes at 37° C., medium was aspirated, ice-cold 0.1 M formic acid in methanol (1 ml) was added and plates were placed at −20° C. overnight. Samples were transferred to tubes using two 2 ml water washes and [³H]-IP was adsorbed to 0.25 ml AG1×8 slurry (formate form, Bio-Rad, Hercules, Calif.), which was washed five times in 5 mM myo-inositol (5 ml) and eluted in 0.75 ml 1.5 M ammonium formate, 0.1 M formic acid. Scintillation counting was performed on 0.5 ml eluate in 5 ml Ecoscint™ (National Diagnostics, Atlanta, Ga.).

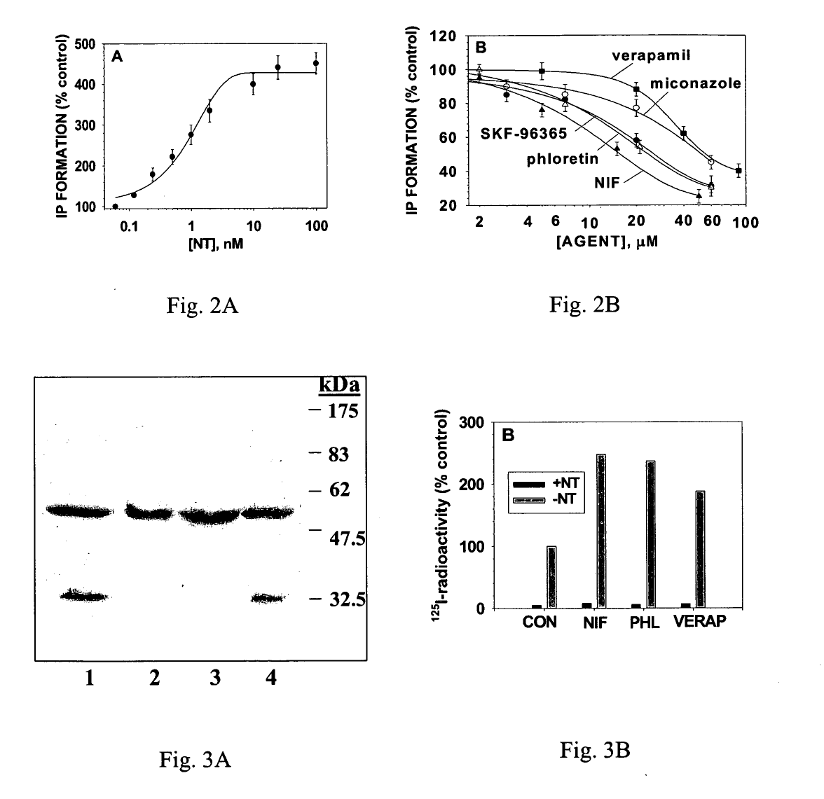

NT increased IP formation≅5-fold in PC3 cells with an EC≅1 nM as shown in FIG. 2A. VGCC blockers inhibited the response to a maximal dose of NT as shown in FIG. 2B, with an efficacy order (NIF>phloretin>verapamil) similar to that for enhancement of NT binding (Table 2). SOCC blockers also inhibited the response to NT (FIG. 2B), giving an efficacy order (SKF-96365>miconazole>trifluoperazine) similar to that for enhancing NT binding (Table 2). For each of these agents, the EC50 for enhancing NT binding was similar to the IC50 for inhibiting NT-induced IP formation (Table 2), and there was a strong statistical correlation (r²=0.842). Enhanced NT-binding and decreased IP formation appear to be linked, e.g., one effect could have caused the other. The results indicated that the effects of CCBs on NT binding and NT-induced IP formation have a similar chemical sensitivity. Although the responses in the two NT assays were correlated, it still was not clear whether these effects depended on the ability of CCBs to block calcium entry into the cells or if some other property of these compounds brought about the effects.

### Example 4

### CCBs Enhanced Photoaffinity Labeling of NTR1 in PC3 Cells

To demonstrate the involvement of NT receptors in the enhanced cellular binding of NT, we measured the effects of CCBs on the labeling of NTR1 isolated by specific immunoprecipitation. The labeled proteins were also identified by western blotting.

For immunoblots, PC3 cells in 60 mm dishes were washed in Locke-containing protease inhibitors: 0.5 mM EDTA, 0.5 μM PMSF, 0.5 μM TPCK and 0.5 μM o-phenanthroline. Protease inhibitors and chemicals whose source is not indicated were obtained from Sigma. Cells were lysed in 100 μl of 2× sodium dodecyl sulfate (SDS) loading buffer with inhibitors, scraped into microfuge tubes and sonicated (20 seconds) on ice. Membranes were isolated from regions of adult rat brains (Carraway et al., Peptides, 14:37-45,1993) and P2 pellets were extracted in 2×SDS loading buffer and sonicated. Cell and tissue extracts were boiled 5 minutes and separated by SDS-PAGE on 10% polyacrylamide minigels. Proteins were electroeluted onto PVDF membranes (Immobilon™ P, Millipore). After blocking in 5% non-fat milk in TTBS: 0.05% Tween™ 20, 20 mm Tris, 0.5 M NaCl, for 1 hour and washing 3 times with TTBS, blots were incubated with the primary antiserum (1:1000) in blocking buffer for 18 hours at 4° C. Our rabbit antiserum (Ab-NTR1) was raised using a synthetic peptide corresponding to residues 398-418 of human NTR1 conjugated to keyhole limpet hemocyanin. The antibodies were affinity purified before use. Blots were washed in TTBS, then incubated with horseradish peroxidase-linked goat anti-rabbit antibody (1:1000) for 1 hour at 20° C., and washed again in TTBS. Enhanced chemiluminescence was performed according to manufacturer (Santa Cruz). After stripping (1 hour at 70° C. in 62.5 mM Tris-HCl, 2% SDS, 0.1 M β-mercaptoethanol, pH 6.8) and washing in TBS, blots were reprobed with antigen-adsorbed antisera to validate the results.

For UV crosslinking: [4-azido-Phe⁶]-NT was iodinated and purified by HPLC to≅1500 Ci/mmol. PC3 cells in 10 cm dishes were incubated with 0.3×10⁶ cpm/ml ¹²⁵I[4-azido-Phe⁶]-NT in 8 ml Locke, 25 minutes at 37° C., in presence and absence of Ca²⁺-channel agents. 1 μM NT was added to controls. Cells were placed on ice for 30 minutes, irradiated at 254 nm with a handheld UV light for 5 minutes at 3 cm, washed in ice-cold phosphate-buffered saline (PBS) and lysed in 10 mM Hepes, 1 mM EDTA, 0.5 mM o-phenanthroline, phenylmethyl sulfonylfluoride (PMSF), and tosyl-phenylalanine chloromethyl ketone (TPCK) (pH 7.4). Membranes, collected by centrifugation (at 30,000 g, 20 minutes) were solubilized in 250 μl 50 mM Tris buffer (pH 7.4), 150 mM NaCl, 0.5% Triton™ X-100, 0.5% NP-40, 5% glycerol at 4° C. for 2 hours. Solubilized NTR1, diluted 2-fold in buffer without detergent, was immunoprecipitated by addition of our rabbit antiserum (Ab-NTR1) (final 1:100). During western blotting, Ab-NTR1 detected two major bands in extracts of PC3 cells, the parent protein of 50 kDa and a breakdown product of 33 kDa, similar to results observed in other cells (Boudin et al., Biochem. J, 305:277-83, 1995). After 18 hours at 4° C., protein A-agarose (10 mg, Sigma) was added for 6 hours. After agarose beads were washed three times with PBS at 4° C., associated radioactivity was measured using a γ-counter. Usually the immunoprecipitate contained≅5% of the solubilized cpm for samples prepared in the absence of NT. SDS-PAGE was used in some cases to validate that the radiolabeled material represented NTR1. For this, the beads were boiled 5 minutes in an equal volume of 2×SDS sample buffer and extracts were subjected to SDS-PAGE using 10% polyacylamide gels, followed by autoradiography.

NTR1 is a 46 kDa protein that has been immunologically characterized and labeled using UV-activatable crosslinkers (Mazella et al., J. Biol. Chem., 263:144-49, 1988). Initially, we used western blotting to verify the specificity of our antiserum (Ab-NTR1) raised towards the C-terminus of human NTR1. Whereas extracts of rat brain gave a single band at ≅50 kDa, PC3 cells gave this parent protein, along with a 33 kDa fragment (FIG. 3A), in keeping with published results (Boudin et al., Biochem. J., 305:277-283, 1995). Next, we used UV-light to incorporate ¹²⁵I-(4-azido-Phe⁶)-NT into PC3 cells treated with CCBs or control, and we assessed the incorporation of radioactivity into immunoprecipitated NTR1. The results in FIG. 5B showed that the radioactivity associated with NTR1 was enhanced by NIF (2.8 fold; p<0.001), phloretin (1.8 fold; p<0.05) and verapamil (1.5 fold; p<0.05) as compared to the control. For each agent, the increase in immunoprecipitated radioactivity (FIG. 3B) was similar to the increase in NT-binding to PC3 cells seen at the appropriate dose (See FIG. 1C). SDS-PAGE and autoradiography on selected samples verified the presence of 50 kDa and 33 kDa radiolabeled proteins (data not shown). These results indicate that the CCBs shown to enhance NT binding did so by increasing the association of ¹²⁵I-NT with NTR1.

### Example 5

### CCBs Had an Indirect Action on NTR1

Since there was precedent in the literature for tyrosine kinase inhibitors acting directly on the EGFR to elevate its binding (Lichtner et al., Cancer Res., 61:5790-5795, 2001), we tested whether CCBs would directly affect the binding of ¹²⁵I-NT to isolated PC3 cell membranes in vitro. PC3 cell membranes were prepared and collected by centrifugation at 30,000 g (Seethalakshmi et al., The Prostate, 31:183-192, 1997). Binding of ¹²⁵I-NT (10⁵ cpm) to membranes (10-50 μg) was performed at 20° C. for 60 minutes in 10 mM Tris-HCl (pH 7.5), containing 1 mM MgCl₂, 1 mM dithiothreitol (DTT), 0.1% BSA and protease inhibitors as described. Membranes were collected and washed onto glass fiber (GF-B) filters using a Brandel cell harvester, and the filters were counted using the γ-counter (Carraway et al., Peptides, 14:37-45, 1993).

NT binding to cell membranes was not increased by NIF, phloretin, or verapamil (Table 3), indicating that these agents were unable to act directly on NTR1. Although a key participant in the reaction might have been lost during membrane isolation, it seems more likely that there was a requirement for cellular metabolism and/or architecture. Thus, the increase in NT binding observed in live cells most likely reflected an indirect effect of CCBs, possibly by way of an effect on cellular enzymes or metabolism.

TABLE 3
Effects of CCBs on NT-binding to PC3 Cell Membranes

| | Specific NT-Binding (% control) at Dose of Agent ^{b} | | | |
|---|---|---|---|---|
| Agent ^{a} | 10 μM | 25 μM | 75 μM | 100 μM |
| NIF | 97 ± 5 | | 102 ± 5 | 108 ± 8 |
| nimodipine | 99 ± 2 | 101 ± 2 | 94 ± 4 | |
| phloretin | 110 ± 7 | 105 ± 8 | 97 ± 8 | |
| verapamil | 100 ± 2 | | 101 ± 5 | 103 ± 4 |

^{a} Agents were freshly dissolved in DMSO at 10 mM and diluted into Locke just before use.
^{b} PC3 cell membranes were preincubated 10 minutes with agents or control, and NT binding was performed at 22° C. for 60 minutes. Specific binding was measured in 4-6 experiments and expressed as % control (mean ± SEM). Results for the various agents were not significantly different from control (p > 0.1).

### Example 6

### Cell-surface Binding Versus Internalization

To characterize the effects of CCBs on cellular NT binding, we determined whether cell-surface binding or internalization (or both) were altered. Binding of ¹²⁵I-NT to PC3 cells was performed as described in Example 1. Cell surface binding and internalization of ¹²⁵I-NT were assessed by washing cells at 22° C. for 2 minutes with 0.6 ml 0.2 M acetic acid, 0.5 M NaCl, pH 3.0 (Beaudet et al., Biochem. Pharmacol., 47:43-52, 1994). Binding at 4° C. achieved equilibrium within 3 hours, at which time>90% of the radioactivity was on the cell surface. Binding at 37° C. reached equilibrium in 25 minutes, at which time≅70% of total binding was internalized. To measure rates of internalization for ¹²⁵I-NT prebound to cells, the following procedure was used. ¹²⁵I-NT (10⁵ cpm) was pre-bound to PC3 cells in 24-well plates at 4° C. for 3 hours. After washing the cells three times in ice-cold PBS,>90% of ¹²⁵I-NT was located on cell surface as determined by acid washing. Agents (10 mM in DMSO) were diluted to 50 μM in Locke and incubated with the cells at 37° C. for 5 minutes. The control was 0.5% DMSO. Cell-surface and internalized ¹²⁵I-NT were measured, and percent internalization per minutes was calculated.

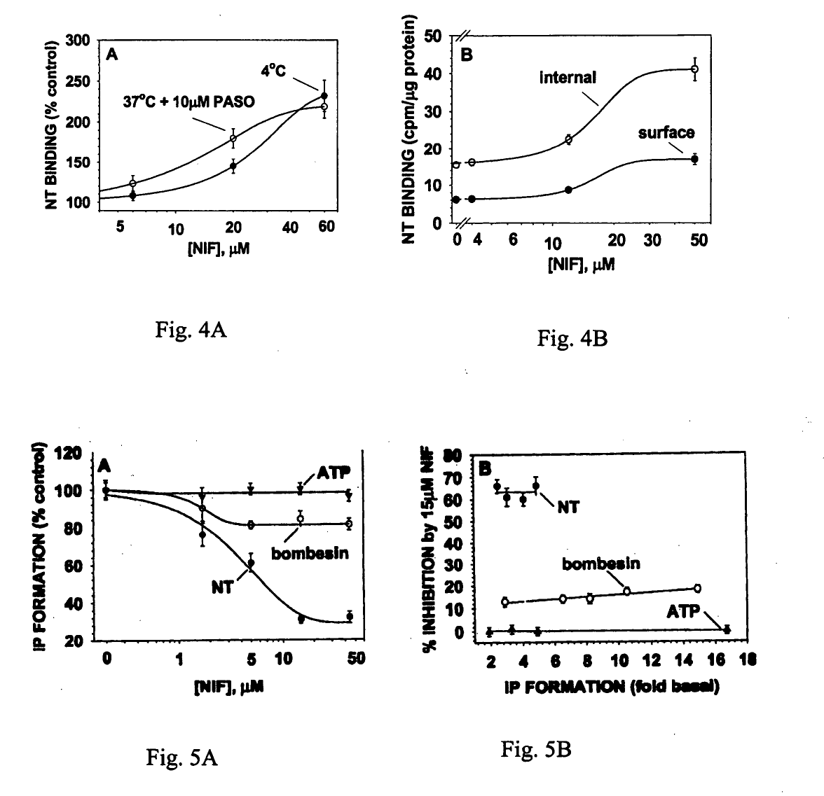

Cell-surface binding of 125I-NT was enhanced by NIF to a similar extent when assessed by three different methods (FIGS. 4A and 4B). NIF increased surface binding 2.4-, 2.2- and 2.7-fold respectively, as measured at 4° C. (FIG. 4A), at 37° C. in the presence of phenylarsine oxide (FIG. 4A), and at 37° C. by acid washing (FIG. 4B). Internalization of ¹²⁵I-NT was 68-72% of total binding in the presence or absence of NIF (FIG. 4B). In addition, the internalization rate at 37° C. for cell-surface 125I-NT, previously bound to cells at 4° C. in the absence of drugs, was unaffected by 50 μM NIF, 50 μM phloretin and 50 μM verapamil. Internalization rates (%/minute; n=12 from two experiments) were: control, 8.6±0.6; NIF, 8.0±0.6; phloretin, 8.1±0.7; verapamil, 9.2±0.7, which did not differ significantly (p>0.1). The results indicated that these agents increased cellular NT binding by enhancing the interaction of NT with NTR1, rather than by enhancing the internalization rate for the NT-NTR1 complex.

### Example 7

### Enhancement of Cellular NT-Binding by CCBs was Specific to NT

To determine whether the effects of CCBs on NT-binding to PC3 cells were receptor-specific, we measured the effects on cellular binding of ligands specific for other G-protein receptors (GPCRs) and for EGF-receptor (EGFR). Radioreceptor assays using the labeled ligands of Example 1 were developed for β2-adrenergic, bombesin, EGF and V₁ₐ-vasopressin receptors. Table 4 shows the ligands used and the binding parameters determined. Assessing the effects of CCBs, we found that NIF, phloretin, verapamil and SKF-96365 did not enhance β2-adrenergic, V₁ₐ-vasopressin and EGF receptor binding to PC3 cells (Table 4). However, bombesin receptor binding was elevated slightly (≅19%) by NIF (Table 4). β2-adrenergic receptor binding was decreased by these agents (Table 4), although this was due to a direct competition with ¹²⁵I-pindolol (as indicated by the structural resemblance of the agents to pindolol and the fact that ¹²⁵I-pindolol binding to PC3 cell membranes was inhibited similarly (see Table 4 footnote). Cell binding for the vasopressin receptor was also decreased by these drugs (Table 4) (see Table 4, footnote). Overall, these data indicated that the robust elevation in ligand binding to PC3 cells caused by CCBs was specific to the NT receptor, although the bombesin receptor could also respond to a lesser degree.

TABLE 4
Effects of CCBs on PC3 Cell-binding of Ligands Specific for
Bombesin-, Vasopressin-, β2-adrenergic- and EGF-receptors

| | | Specific Binding (% control) at Dose | |
|---|---|---|---|
| | | of Agent ^{a} | |
| Ligand | Agent | 12 μM | 60 μM |
| ¹²⁵I-[Nle¹⁴]- | NIF | 108 ± 4 | 119 ± 4** |
| bombesin | phloretin | 104 ± 3 | 111 ± 4 |
| | verapamil | 104 ± 4 | 104 ± 4 |
| | SKF-96365 | 99 ± 2 | 106 ± 4 |
| ¹²⁵I-Pindolol^{b} | NIF | 105 ± 5 | 82 ± 3** |
| | Phloretin | 102 ± 4 | 93 ± 3 |
| | Verapamil | 68 ± 5** | 35 ± 6** |
| | SKF-96365 | 86 ± 2** | 51 ± 2** |
| ¹²⁵I-HOLVA^{c} | NIF | 95 ± 4 | 59 ± 5** |
| | Phloretin | 92 ± 4 | 73 ± 4** |
| | Verapamil | 85 ± 4* | 58 ± 4** |
| | SKF-96365 | 80 ± 2** | 50 ± 2** |
| ¹²⁵I-EGF | NIF | 100 ± 4 | 108 ± 4 |
| | Phloretin | 98 ± 2 | 95 ± 4 |
| | Verapamil | 103 ± 4 | 96 ± 4 |
| | SKF-96365 | 103 ± 3 | 94 ± 3 |

^{a} Specific binding of each ¹²⁵I-ligand was measured to PC3 cells. Binding was expressed as % control (mean ± SEM) for 3 to 6 independent experiments.
^{b} Verapamil and SKF-96365 resemble pindolol structurally. Thus, the decrease in binding was due to direct competition with the ligand (% crossreaction, ≅0.0005). This conclusion was supported by the fact that these agents also inhibited the binding of ¹²⁵I-pindolol to PC3 cell membranes (see Methods and Table 3). Binding (% control ± SEM) for 3 experiments in duplicate was: 60 μM verapamil (9 ± 2); 60 μM SKF-96365 (18 ± 5).
^{c} These agents did not resemble HOLVA structurally and they did not inhibit the binding of ¹²⁵I-HOLVA to PC3 cell membranes. Binding (% control ± SEM) for 3 experiments in duplicate was: 60 μM verapamil (91 ± 5); 60 μM SKF-96365 (110 ± 3); 60 μM NIF (90 ± 3).
*Result was significantly different from control (p < 0.05).
**Result was significantly different from control (p < 0.01).

### Example 8

### Inhibition of IP Formation by CCBs was Specific to NT

To examine receptor specificity, we tested the ability of NIF to inhibit IP formation in response to G protein receptor (GPCR) agonists known to stimulate PLC. IP formation was performed as described in Example 3. Preliminary dose-response experiments showed that a maximal dose of NT (30 nM), bombesin (20 nM) and ATP (10 μM) stimulated IP formation by ≅5-fold, ≅15-fold and ≅17-fold, respectively. When PC3 cells were pretreated with varying amounts of NIF, we found that the response to this dose of NT was inhibited as much as ≅69%, whereas that for bombesin was inhibited≅19%, and that for ATP was not inhibited (FIG. 5A). When the dose of each agonist was varied, we found that the % inhibition by 15 μM NIF was independent of the level of stimulation. Thus, at each dose, the response to NT was inhibited≅64%, whereas that for bombesin was inhibited≅15%, and that for ATP was not inhibited (FIG. 5B). These results indicated that the robust inhibition of IP formation by NIF was specific to NT, although the response to bombesin was also inhibited to a lesser degree.

### Example 9

### DHP Inhibited NT-mediated Influx of ⁴⁵Ca²⁺ into PC3 Cells

To determine whether CCBs altered the movement of calcium into PC3 cells, the method of Katsura et al., Mol. Brain Res., 80:132-141 (2000) was used to measure ⁴⁵Ca²⁺ influx in response to NT. Briefly, confluent PC3 cells in 24-well dishes were washed with Ca²⁺-free Locke and pretreated for 10 minutes with 0-36 μM NIF (600 μl per well). The reaction was initiated by addition of 2001 μl NT, followed in 2 minutes by 2.5 mM CaCl₂ (5 μCi ⁴⁵Ca²⁺ per well). After 8 minutes, the cells were washed three times with ice-cold Locke and solubilized in 0.25 M NaOH. The cell extract was neutralized with acetic acid and scintillation counting was performed to measure ⁴⁵Ca²⁺ radioactivity.

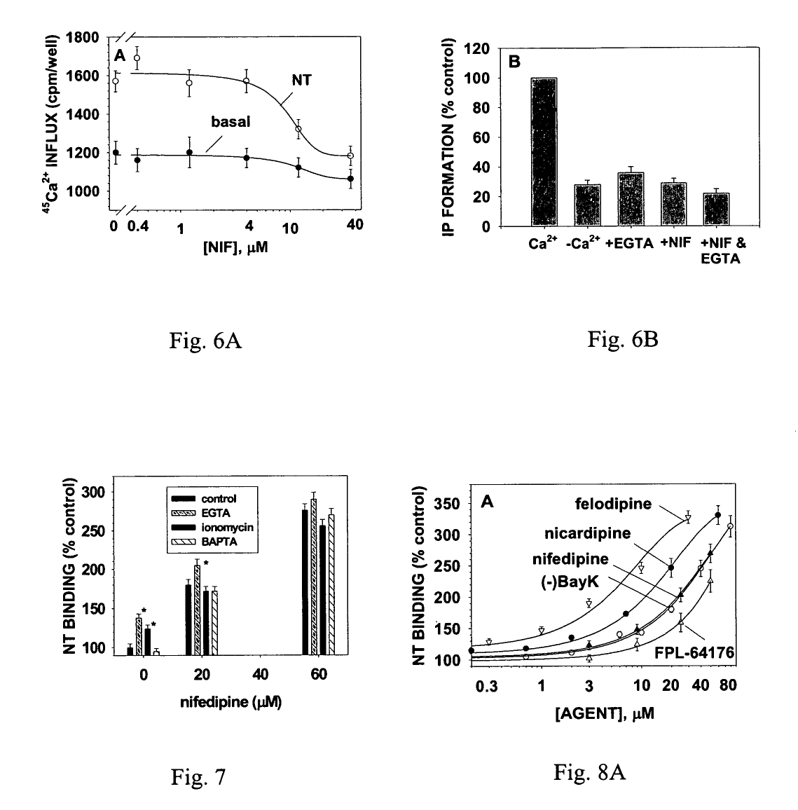

NT enhanced the influx of ⁴⁵Ca²⁺ into PC3 cells, giving an EC50 (≅1 nM) similar to that for NT-induced IP formation. At doses shown to enhance NT binding (FIG. 1C) and to inhibit NT-induced IP formation (FIG. 2B), NIF inhibited the influx of ⁴⁵Ca²⁺ in response to NT (FIG. 6A). These results indicate that the effects of NIF on NT binding and IP-formation involved changes in calcium movement.

### Example 10

### Ca²⁺-dependence of NT-induced IP Formation

Since NT caused an influx of ⁴⁵Ca²⁺ in PC3 cells that was inhibited by NIF, it was possible that CCBs altered NT function by blocking Ca²⁺-influx. Since the enzyme that mediates IP formation (phospholipase C) was Ca²⁺-dependent and since NIF blocked NT-induced IP formation and Ca²⁺-influx at the same dose, we hypothesized that Ca²⁺-influx was required for the IP response and that CCBs blocked this step. IP formation in response to NT was assessed as in Example 3. NT-induced IP formation was inhibited by omitting Ca²⁺ from the buffer, by adding Ca²⁺-chelator EGTA, or by adding NIF (FIG. 6B). Paradoxically, the removal of Ca²⁺ elevated basal IP production≅2-fold, perhaps by mobilizing internal Ca²⁺-stores. However, inhibition of the NT response was not due to a ceiling effect, since IP formation could be elevated>15-fold by bombesin and ATP.

Ca²⁺-ionophore (ionomycin) stimulated IP formation, reproducing≅63% of the NT response. IP formation (% control) was: 2 μM ionomycin, 139±6% (p<0.01); 20 μM ionomycin, 324±14% (p<0.01); 30 nM NT, 457±12% (p<0.01). When added 2 minutes after a maximal dose of NT (30 nM), low doses of ionomycin (2-10 μM) enhanced the NT response. IP formation (% control) was: 10 μM ionomycin, 157±5 (p<0.01); NT, 366±20 (p<0.01), NT plus ionomycin, 465±9 (p<0.001). In contrast, a maximal dose of ionomycin gave less than additive enhancement of the NT response. IP formation (% control) was: (25 μM ionomycin, 322±11 (p<0.001); NT, 384±14 (p<0.001); NT plus ionomycin, 476±15 (p<0.001).

These data suggest that the inhibition of NT-induced IP formation by CCBs were at least partly attributable to a change in Ca²⁺-influx.

### Example 11

### NT Binding to PC3 Cells was Largely Ca²⁺-Independent

To test the hypothesis that CCBs (such as NIF) altered NT binding by inhibiting Ca²⁺-movement, the effects of Ca²⁺-chelators and Ca²⁺-ionophores on NIF-mediated enhancement of NT binding to PC3 cells were examined. Ca²⁺-chelators ethylene glycol tetra-acetic acid (EGTA), and 1,2-bis(o-aminophenoxy)ethane-N,N,N′,N′-tetraacetic acid tetra(acetoxymethyl) ester (BAPTA-AM) were from Sigma, and Ca²⁺-ionophore ionomycin was from Calbiochem. Blocking Ca²⁺-influx with 2 mM EGTA enhanced NT binding (38±6% increase; p<0.05) but the effect was small relative to the 200% increase by NIF. In addition, the ability of NIF to enhance NT binding persisted in the absence of extracellular Ca²⁺. Enhancement of NT binding by NIF was not reversed by 20 μM ionomycin, nor was NT binding altered by chelation of intracellular Ca²⁺ using 50 μM BAPTA-AM. Data are shown in FIG. 7.

In contrast to the results of Example 10, where the effect of NIF on NT-induced IP formation was Ca²⁺-dependent, these data indicate that the effect of NIF on NT binding was Ca²⁺-independent.

### Example 12

### Ca²⁺-Channel Agonists and Antagonists Both Enhance NT Binding

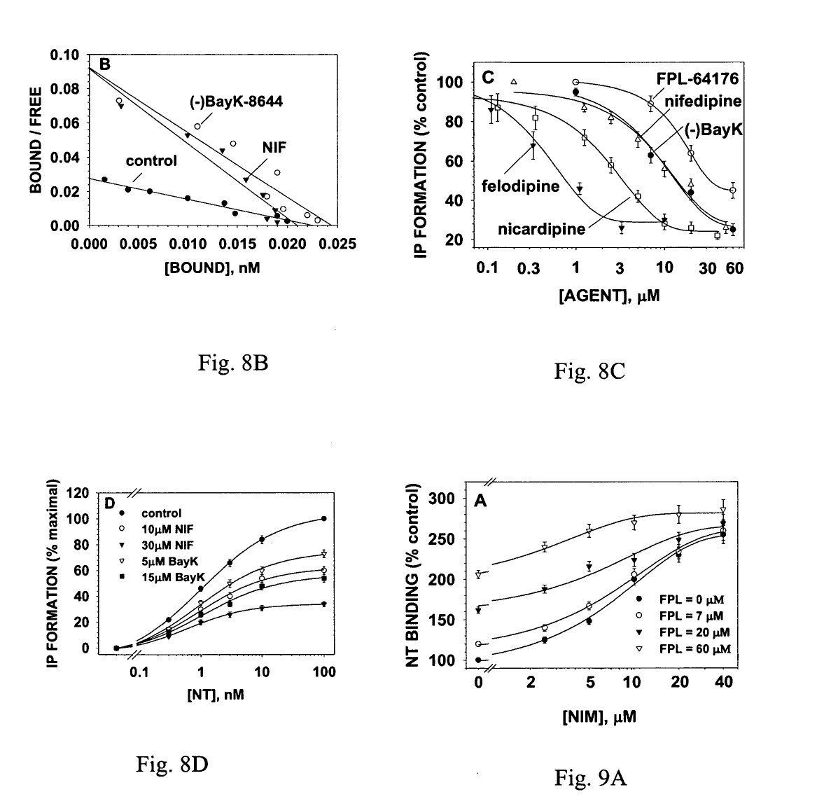

To further test the importance of Ca²⁺ for the effects of CCBs on NT binding and IP formation, experiments involving Ca²⁺-channel agonists and antagonists were performed using methods described in Examples 1 and 3. The results in FIG. 8A show that the VGCC agonist (−) BayK-8644 and the antagonist NIF enhanced NT binding to a similar extent. Both compounds enhanced binding by increasing NTR1 affinity, not by altering receptor number (FIG. 8B; Table 1). Another VGCC agonist FPL-64176 (Biomol, Plymouth Meeting, PA), known to act at a unique non-DHP site (Zheng et al., Mol. Pharmacol., 40:734-41, 1991), was also active, although less potent (FIG. 8A). In addition, the agonists (−) BayK-8644 and FPL-64176 shared with the antagonist NIF an ability to inhibit NT-induced IP formation (FIG. 8C). For each agent, the NT dose-response relationship was shifted downward, indicating that the efficacy of NT was decreased, not its potency (FIG. 8D).

These results were consistent with the possible involvement of SOCC, but not VGCC, in the effects of CCBs on NTR1 function. However, this would only apply to IP formation, not to NT binding. Since IP formation was Ca²⁺-dependent, the fact that these agents decreased the efficacy of NT was in keeping with their known ability to diminish SOCC conductance. However, the simplest idea was that some other property of these drugs (not involving Ca²⁺) accounted for the effects of CCBs on NTR1 function.

### Example 13

### Agents in Combination Gave Additive Effects

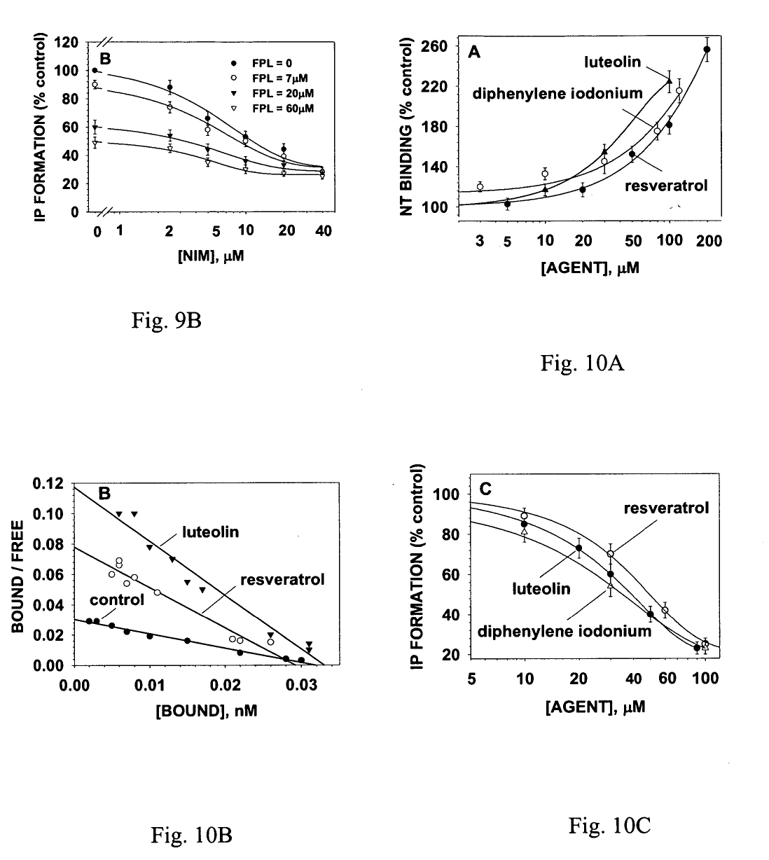

To further test the importance of Ca²⁺-movement, dose-response studies were performed using combinations of Ca²⁺-channel antagonist NIM and agonist FPL-64176, which are known to bind at distinct, discrete sites on VGCC. FIG. 9A shows that NT binding was enhanced in an additive manner at low concentrations of each drug, although the results were less than additive at high concentrations.

FIG. 9B shows that NT-induced IP formation was inhibited in an analogous manner and at high concentrations, it reached a limit at ≅70% inhibition. Similar studies were performed using various combinations of NIF, verapamil, and diltiazem (all antagonists). Again, when low doses of these drugs were combined, additive effects were observed for the enhancement of NT binding and for the inhibition of NT-induced IP formation, whereas at high doses the effects were less than additive. No potentiative or antagonistic effects were observed.

Taken together, these results indicate that the drugs tested, whether Ca²⁺-channel agonists or antagonists, acted in a similar and additive manner to alter NTR1 function.

### Example 14

### Effects on NTR1 Function Correlate with Antioxidant Activity

The most potent and efficacious CCBs to alter NT binding and IP formation were DHPs (such as nifedipine, nimodipine). Because DHPs are known to exhibit antioxidant ability (Mak et al., Pharmacol. Res., 45:27-33, 2002), the relationship between their antioxidant activity and their ability to affect NTR1 function was investigated. DHPs were reported to inhibit Fe³⁺/ascorbate stimulated lipid peroxidation in rat brain slices with activity order: nicardipine>nimodipine>nifedipine (Diaz-Araya et al., Gen. Pharmacol., 31:385-91, 1998). The same activity order was found when these agents were compared for ability to (a) enhance NT binding and (b) inhibit NT-induced IP formation. In both systems, nicardipine was 2- to 4-fold more potent than nifedipine (Table 5). Felodipine was 2-to 4-fold more active than nicardipine (Table 5). While felodipine was reported to be inactive in the rat brain assay mentioned above, it was reported to be more active than nicardipine in a similar antioxidant assay using myocardial membranes (Janero and Burghardt, Biochem. Pharmacol., 38:4344-4348, 1989).

The relative chemical reactivity of DHPs with superoxide anion was reported to be: felodipine>nimodipine>nifedipine>compound-1 (Ortiz et al., Pharm. Res., 20:292-296, 2003). For these substances, the potency to alter NT binding correlated to antioxidant activity, giving r²=0.89 (Table 5). Compound-1, a DHP analog with N-ethyl in place of the NH moiety, was reported by Ortiz et al. (supra) to have a greatly reduced reactivity with superoxide (<10% that of felodipine). Here, we found that it displayed≅4% the activity of felodipine and 20-50% the activity of nifedipine in altering NTR function (Table 5). These results indicate that DHPs enhance NT-binding and reduce NT-mediated IP formation, by reaction(s) involving hydrogen donation, i.e., antioxidant activity.

TABLE 5
Activity of DHPs and Polyphenols
on NT Binding and IP-Formation

| | | NT Binding ^{a} | IP | Antioxidant ^{c} |
|---|---|---|---|---|
| Classifi- | | EC50 | Formation ^{b} | Activity |
| cation | Agent | (μM) | IC50 (μM) | (relative) |
| VGCC | felodipine | 3 | 1 | 2.86 |
| antagonist | nitrendipine | 7 | 2 | 1.34 |
| | nicardipine | 7 | 3 | |
| | nimodipine | 7 | 6 | 2.12 |
| | nifedipine | 15 | 15 | 0.73 |
| | compound-1 ^{d} | 75 | 28 | <0.30 |
| VGCC | BayK-8644 | 16 | 15 | |
| agonist | FPL-64176 | 29 | 27 | |
| Anti- | luteolin | 40 | 38 | |
| oxidant | resveratrol | 80 | 48 | |
| | Diphenylene | 61 | 35 | |
| | iodonium | | | |
| | BHA | 110 | nd | |

^{a} EC50 was defined as the [agent] giving 75% increase in NT binding. The data are means determined in at least 3 experiments.
^{b} IC50 was defined as the [agent] giving 50% decrease in IP formation. The data are means from at least 3 experiments. nd, not determined.
^{c} Relative activity coefficient for reactivity to superoxide ion. Data from Ortiz et al., 2003.
^{d} Compound-1 (Ortiz et al., supra, 2003) is an N-ethyl DHP (structure in FIG. 7) that displays reduced reactivity to superoxide. Its effects on calcium channels are not known.

### Example 15

### Effects of Other Antioxidants

ROS scavengers include vitamin-like antioxidants, flavonoids and polyphenols (Rice-Evans et al., Free Radic Biol Med., 20:933-956, 1996). Testing vitamin-like antioxidants (obtained from Sigma) on NT binding in PC3 cells, we found β-carotene, thiamine, riboflavin, pyridoxine, ascorbic acid, α-tocoferol, and tetrahydro-biopterin to be ineffective (used at 20-180 μM; n=3), while vitamin K (menadione) had a small effect at 180 μM (% control: 168±8; n=3; p<0.05). Other antioxidants without effect included N-acetyl cysteine, glutathione, and sodium borohydride (used at 1-3 mM; n=3); trolox, ellagic acid, (+)-catechin, (−)-epigallocatechin gallate, and rutin (used at 10-100 μM; n=3).

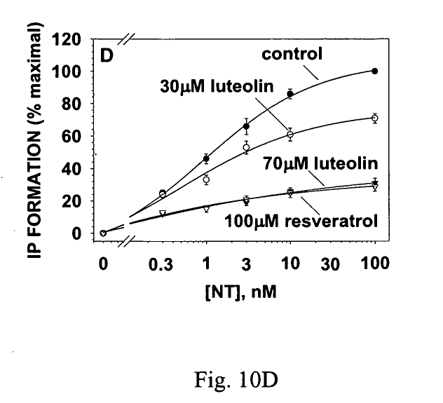

In striking contrast were the results for the polyphenolic antioxidants (obtained from Calbiochem), namely luteolin (a flavonoid) and resveratrol, which displayed effects that were indistinguishable from those of DHPs. Luteolin and resveratrol enhanced NT binding (FIG. 10A), and the effect involved an increase in NTR1 affinity without a significant change in NTR1 number (FIG. 10B; Table 6). Luteolin and resveratrol also inhibited NT-induced IP formation (FIG. 10C), and the effect involved a dose-dependent decrease in NT efficacy (FIG. 10D). When tested together for effects on NT binding, the response to luteolin plus nimodipine and resveratrol plus nimodipine were additive at low doses of each agent, while they were less than additive at high doses. The data demonstrate that polyphenolic antioxidants mimicked the effects of DHPs and appeared to act via the same pathway.

TABLE 6
Effects of CCBs and Antioxidants on
NT Binding Parameters in PC3 Cells

| Agent ^{a} | | Classification | Bmax ^{b} (fmol/mg) | Ki ^{b} (nM) |
|---|---|---|---|---|
| none | | control | 155 ± 11 | 1.0 ± 0.07 |
| 50 | μM | VGCC | 152 ± 10 | 0.5 ± 10.05^{c} |
| | nifedipine | antagonist | | |
| 50 | μM BayK- | VGCC | 162 ± 12 | 0.56 ± 0.06^{c} |
| | 8644 | agonist | | |
| 60 | μM | flavonoid | 164 ± 10 | 0.62 ± 0.05^{c} |
| | luteolin | antioxidant | | |
| 150 | μM | polyphenol | 171 ± 11 | 0.36 ± 0.04^{c} |
| | resveratrol | antioxidant | | |

^{a} PC3 cells were pretreated 10 minutes with indicated concentrations of each agent or vehicle control. ¹²⁵I-NT (10⁵ cpm, 50 pM) was added and specific binding was measured at 37° C.
^{b} Scatchard analyses were performed using 12 concentrations of NT. The results for Bmax and Ki (mean ± SEM) were from at least 3 experiments per agent.
^{c} Indicates significant difference (p < 0.05) as compared to control.

### Example 16

### Effect of DHPs on NTR1 Does Not Involve Sulfhydryl Groups

Since some antioxidants act by reducing sulfhydryl groups on proteins, we tested the hypothesis that DHPs increase NT binding by maintaining sulffiydryl group(s) in a reduced state. Confirming the importance of sulfhydryl groups, we showed that sulfhydryl chelators, Ni³⁺ (IC₅₀, ≅50 μM) and Cd²⁺ (IC₅₀, ≅600 μM), inhibited NT binding to PC3 cells, and that their effects were inhibited by 2 mM DTT.

However, in the basal state, the sulfhydryl group(s) required for NT binding to PC3 cells were primarily reduced, since NT-binding was increased only slightly by 1 mM ascorbic acid (114±7%, n=4), 2 mM DTT (125±8%, n=4) and 5 mM N-acetyl-cysteine (107±6%, n=4). 2 mM DTT did not alter the NT displacement curve and did not inhibit the effects of nifedipine. NT binding was enhanced similarly by 50 μM nifedipine in the presence and absence of 2 mM DTT (control, 246±10%; DTT, 231±11%; n=4). NT-induced IP-formation was inhibited similarly by nifedipine in the presence and absence of DTT. Thus, DHPs acted by an antioxidant mechanism that did not involve the reduction of sulfhlydryl groups in NTR1.

### Example 17

### Effect of DHPs on NTR1 Involves Flavoprotein Dehydrogenase(s)

Diphenylene iodonium (DPI) is an inhibitor of flavoprotein dehydrogenases, enzymes that produce reactive oxygen species (ROS). To test whether DHPs scavenge ROS produced by flavoprotein dehydrogenases, the effect of DPI on NT binding to PC3 cells was tested.

DPI mimicked the effects of DHPs on NT binding (shown in FIG. 10A) and NT-induced IP formation (shown in FIG. 10C). The hydroxy radical scavenger butylated hydroxy anisole (BHA) was also effective (Table 5). These results suggest that flavoprotein dehydrogenases and/or ROS species produced by these enzymes participate in the effects of DHPs on NTR1 function.

### Example 18

### Comparisons of Chemical Structures of DHPs and Polyphenols

As can be seen in FIG. 11, the chemical structures of DHPs and polyphenols are similar, each possessing aromatic ring structures with redox capability. The order of potency (Table 5) for ability to alter NTR1 function (felodipine>nitrendipine≅nicardipine>nimodipine>nifedipine>luteolin>resveratrol) appeared to relate to donor group acidity (NH>OH) and to the number of conjugated double bonds. For DHPs, chloro substituents in the adjacent phenyl ring gave the highest activity (felodipine), while nitro in the meta position was less effective (nitrendipine, nicardipine, nimodipine) and nitro in the ortho position was least effective (nifedipine). Luteolin and resveratrol contained conjugated π-bonded rings which could potentially support the stability of radicals and cations (Solomons, Chapter 15, in Fundamentals of Organic Chemistry, pp. 599-641, 4th ed., John Wiley & Sons, NY 1994). By donating hydrogen(s), DHPs could conceivably form pyridinium or pyridine analogs with an even greater number of conjugated double bonds and potential to support radical and cation formation. The very high membrane partition coefficients displayed by DHPs (Mason et al., Biophys. J, 56:1193-201, 1989) could determine their ability to accumulate at target site(s).

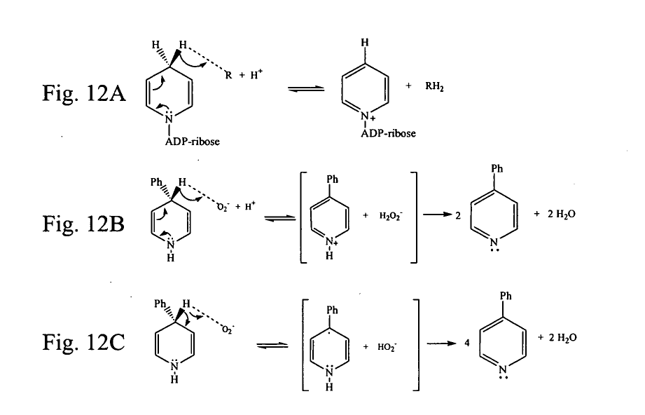

The reaction scheme whereby NAD-linked dehydrogenases donate hydrogen atoms to substrates is shown in FIG. 12A. One hydrogen is transferred from NADH as a hydride ion (H⁻) and another is taken as H⁺ from the medium (Lehninger, Chapter 17, in Principles of Biochemistry, pp. 477, Anderson et al., eds), Worth Publishers, New York, 1982). While not intending to be bound by theory, it is possible that DHPs can react analogously, transferring hydrogen atoms to superoxide by way of cationic (FIG. 12B) or radical intermediates (FIG. 12C) to generate pyridine derivatives and water. DHPs are known to form pyridine adducts when reacted with alkyl radicals (Nunez-Vergara et al., Free Rad. Res., 37:109-120, 2003). Since the stability of the intermediates in FIGS. 16A-16C is negatively affected by electron withdrawal, this predicts that nitro groups in the phenyl ring (especially ortho) would diminish reactivity. The order derived from such considerations (felodipine>nitrendipine≅nicardipine≅nimodipine>nifedipine) is in fair agreement with that measured by Ortiz et al., Pharm. Res., 20:292-296, 2003, and that found here for altering NTR1 function. Since nitrendipine, nicardipine, and nimodipine each have nitro in the meta position, a near equal reactivity with superoxide is expected. The differences in described activity among some antioxidants in our system can be, at least partially, attributed to the effects of ring substituents on lipophilicity (FIG. 11), which affects their ability to enter cells and partition into membranes.

### Example 19

### Lipoxygenase (LOX) Expression in PC3 Cells

To investigate whether other oxidative systems could potentially contribute to the enhancement of NT binding by antioxidants, we assessed the expression of lipoxygenases (LOXs) in PC3 cells. LOXs are lipid-peroxidizing enzymes categorized according to specificity for oxygenation of arachidonic acid; they include 5-LOX, 12-LOX and 15-LOX. Metabolism of arachidonic acid via the LOX pathway has been reported to generate ROS. See, e.g., Nakamura et al., Carcinogenesis, 6:229-235, 1985.

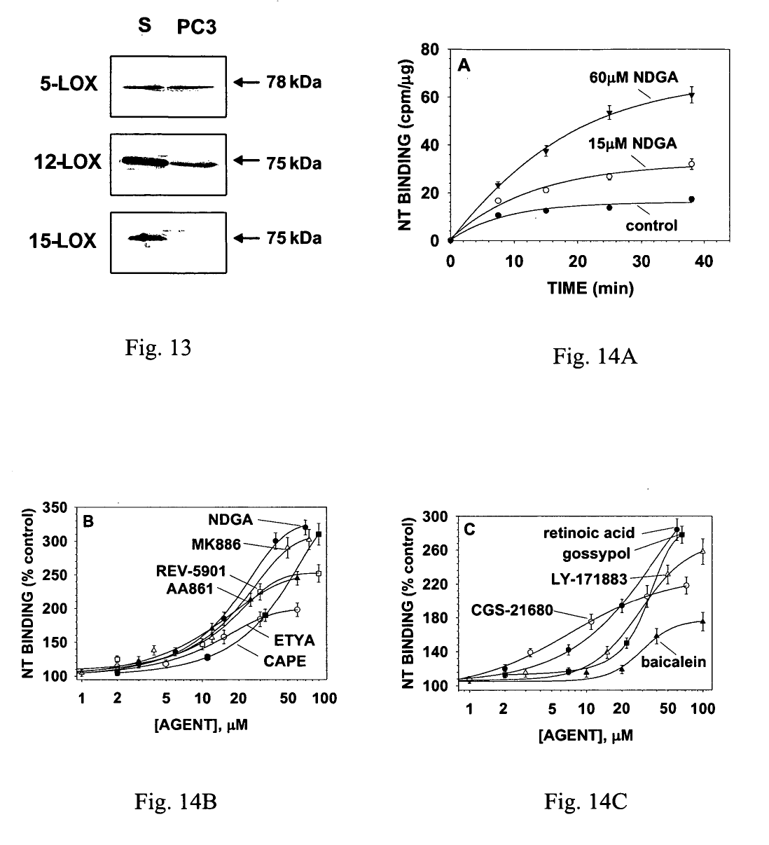

To determine which LOX isoforms might be involved in the regulation of NT binding and signaling, we examined PC3 cell extracts for the presence of these proteins. Western blotting was performed using specific antisera to 5-LOX, 12-LOX and 15-LOX (Cayman Chemical, Ann Arbor, Mich.). LOX isoforms have predicted molecular weights in the range 75-80 kDa (Brash et al., J. Biol. Chem., 274:23679-23682, 1999). As shown in FIG. 13, PC3 cell extracts tested positively for 5-LOX and 12-LOX, giving bands corresponding to the standard. On the other hand, 15-LOX appeared not to be present in PC3 cells. These findings indicate the presence of proteins similar to 5-LOX and 12-LOX in PC3 cells that could be the targets of LOX inhibitor class of antioxidants.

### Example 20

### Antioxidants that are LOX Pathway Blockers Enhance NT Binding

In the following experiments, the antioxidants MK886, LY171883, LY294002, and SB203580 were from Calbiochem (San Diego, Calif.). The antioxidants Rev-5901, AA861, retinoic acid, LY83583, and SQ22536 were from Biomol (Plymouth Meeting, Pa.). The antioxidants Nordihydroguaiaretic acid (NDGA), caffeic acid phenethyl ester (CAPE), gossypol, and 5,8,11,14-eicosatetraynoic acid (ETYA) were from Sigma (St. Louis, Mo.).

Specific binding of ¹²⁵I-NT (10⁵ cpm/ml) to PC3 cells at 37° C. (≅95% of total 125 binding) was ≅16.8±0.81 cpm ¹²⁵I-NT bound/μg protein (n=12), which corresponded to≅3000 cpm ¹²⁵I-NT bound/well. NDGA, a broad specificity LOX inhibitor, dose-dependently increased the apparent rate of and the steady state level of NT binding to PC3 cells (FIG. 14A). Specific binding was enhanced as much as 3-fold, without a change in non-specific binding.

Similar effects were displayed by eleven structurally diverse LOX pathway blockers that acted by a number of different mechanisms. These included four non-selective LOX inhibitors (NDGA, ETYA, CAPE, gossypol), two 5-LOX inhibitors (AA-861, Rev-5901), one FLAP inhibitor (MK886), two inhibitors of leukotriene formation (retinoic acid, CGS-21680), one LTD4 receptor antagonist (LY171883) and one 12-LOX inhibitor (baicalein). NT binding was increased dose-dependently up to 3.2-fold by NDGA, 3.0-fold by MK886, 2.8-fold by CAPE, 2.7-fold by retinoic acid, 2.6-fold by LY-171883, 2.6-fold by gossypol, 2.4-fold by Rev-5901, 2.3-fold by AA-861, 2.2-fold by ETYA, 2.2-fold by CGS-21680 and 1.8-fold bybaicalein (FIGS. 16B and 16C). The specificity of these drugs and the magnitude of their effects on NT binding suggested that the primary target was likely to be 5-LOX, rather than 12-LOX or 15-LOX.

### Example 21

### LOX Pathway Blockers Inhibit NT-induced IP Formation

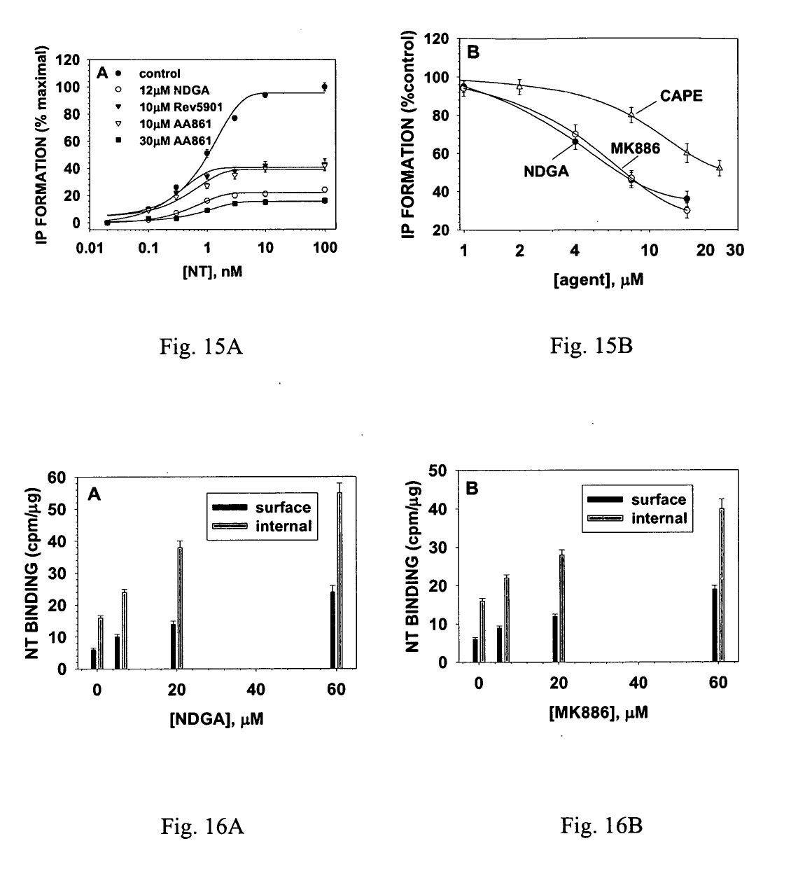

Although antioxidants that are LOX-inhibitors enhanced NT receptor binding, they reduced the ability of NT to stimulate phospholipase C. NT increased IP formation in PC3 cells with an EC50 value of ≅1 nM (FIG. 15A), which was in agreement with the receptor Kd (Carraway et al., J. Pharmacol. Exp. Ther., 307:640-50, 2003). LOX pathway blockers dose-responsively inhibited NT-induced IP formation (e.g., FIG. 15B). The efficacy of NT was reduced, not its potency (FIG. 15A), suggesting that these agents either shifted NTR1 to a non-functional state or they decreased the reaction rate by inhibiting phospholipase C or diminishing substate levels.

For each of the agents examined (Table 7), the IC50 value for inhibiting NT-induced IP formation was related to the EC50 value for enhancing NT binding, and there was a strong statistical correlation (r²=0.94). The data thus indicate that these two responses had a similar chemical sensitivity to the (antioxidant) LOX inhibitor drugs tested and/or that they were linked, e.g., that one led to the other.

TABLE 7
Activity of LOX Blockers on NT Binding and NT-induced IP Formation

| | | NT Binding ^{a} | | IP Formation ^{b} | 5-LOX Activity ^{c} |
|---|---|---|---|---|---|
| | | Efficacy | EC50 | IC50 | IC50 |
| Inhibitor Type | Agent | % increase | μM | μM | μM |
| non-selective | NDGA | 230 | 16 | 7 | 0.3-2 |
| LOX inhibitors | ETYA | 100 | 50 | 32 | 6-50 |
| | CAPE | 220 | 34 | 18 | |
| | Gossypol | 170 | 32 | 20 | |
| 5-LOX Inhibitors | Rev5901 | 160 | 19 | 8 | 10 |
| | AA861 | 155 | 19 | 7 | 0.1-2 |
| FLAP Inhibitor | MK886 | 210 | 18 | 8 | 0.1-1 |
| 12-LOX Inhibitor | Baicalein | 75 | >100 | >100 | |
| Blockers of | Retinoic | 180 | 20 | 7 | |
| Leukotriene | Acid | | | | |
| Formation | CGS21683 | 228 | 22 | nd | 0.01-1 |
| LTD4 receptor | LY171883 | 157 | 34 | nd | |
| Antagonist | | | | | |

^{a} EC50 was defined as the [agent] giving 100% increase in NT binding. Data are means from at least 3 experiments.
^{b} Since the maximal inhibition of IP formation was ≅80%, IC50 was defined as [agent] that decreased IP formation by 40%. Data are means determined in at least 3 experiments.
^{c} IC50 is given as the range of reported values for inhibition of 5-LOX activity measured in various blood cell systems (Walker et al., 2002; Flamand et al., 2000; Ford-Hutchinson et al., 1994; Radmark, 2000; and references therein).

### Example 22

### LOX Pathway Blockers Do Not Act Directly on NTR1

Binding of ¹²⁵I-NT to isolated PC3 cell membranes in vitro was not dramatically increased by NDGA, MK886, Rev-5901, AA-861 and retinoic acid (Table 8), suggesting that LOX-directed agents did not act directly on isolated NTR1. Although a key participant in the reaction might have been lost during membrane isolation, it seems more likely that there was a requirement for cellular metabolism and/or architecture. Thus, the increase in NT binding observed in live cells most likely reflected an indirect effect possibly involving inhibition of ROS production by the LOX pathway (See FIG. 20).

TABLE 8
Effects of LOX Pathway Blockers on
NT-binding to PC3 Cell Membranes

| | Specific NT-Binding (% control) at Dose of Agent ^{a} | | |
|---|---|---|---|
| Agent | 10 μM | 25 μM | 75 μM |
| NDGA | 110 ± 5 | 121 ± 8 | 132 ± 10* |
| MK886 | 99 ± 3 | 107 ± 4 | 97 ± 4 |
| Rev-5901 | 101 ± 4 | 113 ± 5 | 110 ± 5 |
| AA-861 | 107 ± 4 | 109 ± 4 | 110 ± 5 |
| Retinoic acid | 102 ± 3 | 115 ± 4* | 142 ± 8** |

^{a} PC3 cell membranes were preincubated for 10 minutes with agents or control, and NT binding was performed at 22° C. for 60 minutes. Specific binding was measured in 4 experiments and expressed as % control (mean ± SEM).
*Result was significantly different from control (p < 0.05).
**Result was significantly different from control (p < 0.01).

### Example 23

### Receptor Specificity

To determine if the effects were specific to NTR1, we tested LOX pathway blockers for effects on PC3 cell binding of ligands for other receptors using assays previously described above. The results in Table 9 show that that NDGA, MK886, AA861 and Rev-5801 did not have dramatic effects on the binding of ligands for the bombesin, V₁ₐ-vasopressin, β2-adrenergic and EGF receptors. There was only a modest increase (19-35%) in bombesin receptor binding (all agents) and a 65% increase in EGF binding (only MK-886). The 24-34% decrease in β2-adrenergic receptor binding was likely due to structural similarity of the agents to epinephrine. Thus, the robust elevation in cell binding (>200% increase) caused by LOX-directed agents was specific to NTR1.

TABLE 9
Effects of LOX Blockers on PC3 Cell Binding of Ligands Specific
for Bombesin-, Vasopressin-, β2-adrenergic- and EGF-receptors

| | | Specific Binding (% control) at Dose | |
|---|---|---|---|
| | | of Agent ^{e} | |
| Ligand | Agent | 12 μM | 60 μM |
| ¹²⁵I-[Nle14]- | NDGA | 110 ± 3 | 127 ± 4** |
| bombesin ^{a} | MK886 | 106 ± 5 | 125 ± 7* |
| | AA-861 | 112 ± 4 | 135 ± 6** |
| | Rev-5901 | 107 ± 3 | 119 ± 3** |
| ¹²⁵I-HOLVA ^{b} | NDGA | 84 ± 4** | 50 ± 4** |
| | MK886 | 93 ± 5 | 67 ± 3 ** |
| | AA-861 | 87 ± 3* | 57 ± 3** |
| | Rev-5901 | 68 ± 5** | 42 ± 3 ** |
| ¹²⁵I-Pindolol ^{c} | NDGA | 86 ± 4* | 66 ± 9** |
| | MK886 | 105 ± 3 | 102 ± 6 |
| | AA-861 | 107 ± 5 | 102 ± 6 |
| | Rev-5901 | 97 ± 2 | 76 ± 3** |
| ¹²⁵I-EGF ^{d} | NDGA | 114 ± 4 | 112 ± 6 |
| | MK886 | 141 ± 7** | 165 ± 11** |
| | AA-861 | 112 ± 5 | 100 ± 3 |
| | Rev-5901 | 101 ± 3 | 92 ± 5 |

^{a} This ligand for bombesin receptor gave 95% specific binding (Bmax = 1016 ± 64 fmol/mg).
^{b} This ligand for V1ₐ-vasopressin receptor gave 77% specific binding (Bmax = 156 ± 12 fmol/mg). None of the agents resembled HOLVA structurally.
^{c} This ligand for (β2-adrenergic receptor gave 66% specific binding (Bmax = 86 ± 6 fmol/mg). Since NDGA and Rev-5901 resemble epinephrine structurally, the decrease in binding was most likely due to direct competition with the ligand (% crossreaction, ≅0.0005).
^{d} This ligand for EGF-receptor gave 95% specific binding (Bmax = 151 ± 11 fmol/mg).
^{e} Specific binding of each ¹²⁵I-ligand was measured to PC3 cells at 37° C. Binding was expressed as % control (mean ± SEM) for at least three independent experiments.
*Result was significantly different from control (p < 0.05).
**Result was significantly different from control (p < 0.01).

### Example 24

### Specificity for LOX Pathway

NT binding was not dramatically altered by inhibitors of cyclooxygenase (indomethacin, phenbutazone), nitric oxide synthase (L-NAME, L-NMMA), guanylyl cyclase (LY83583), adenylyl cyclase (SQ22536), and various protein kinases (PD98059, LY294002) at concentrations known to affect these enzymes (Table 10). NT binding was also not much affected by general reducing agents (e.g., sodium borohydride, dithiothreitol) and antioxidants (e.g., trolox, ascorbic acid, thiamin, riboflavin, pyridoxine). Inhibitors, chemicals, and antioxidants tested were from Sigma.

These results indicated that the robust effects of LOX inhibitors were not likely to involve non-specific actions or general effects on protein sulfhydryl groups.

TABLE 10
Effects of Various Enzyme Inhibitors on NT Binding to PC3 Cells

| | | Specific NT-Binding (% control) at Dose of Agent ^{b} | | | |
|---|---|---|---|---|---|
| Agent ^{a} | Enzyme | 6 μM | 20 μM | 60 μM | 200 μM |
| indomethacin | cyclooxygenase | 109 ± 4 | 95 ± 6 | 113 ± 7 | |
| phenylbutazone | cyclooxygenase | 113 ± 4 | 100 ± 4 | 89 ± 9 | |
| L-NAME | NO synthase | | 101 ± 3 | 103 ± 4 | 95 ± 6 |
| L-NMMA | NO synthase | | 103 ± 5 | 107 ± 3 | 105 ± 7 |
| LY83583 | guanylyl cyclase | 106 ± 4 | 96 ± 5 | 95 ± 6 | |
| SQ22536 | adenylyl cyclase | 98 ± 3 | 108 ± 4 | 105 ± 4 | |
| PD98059 | MAPK-kinase | 113 ± 7 | 121 ± 7* | 131 ± 9* | |
| U0126 | MAPK-kinase | 118 ± 6 | 108 ± 6 | 120 ± 9 | |
| LY294002 | PI3-kinse | 115 ± 9 | 118 ± 6* | 132 ± 8 | |
| SB203580 | p38 MAP kinase | 108 ± 8 | 116 ± 7 | 125 ± 9 | |

^{a} L-NAME and L-NMMA were dissolved in water and all other agents were in DMSO at 10 mM. Agents were diluted into Locke just before use.
^{b} PC3 cells were preincubated with agent or control vehicle for 10 minutes prior to the NT binding reaction. Specific NT binding is given as % control (mean ± SEM) for at least three independent experiments.
*Results were significantly different from control (p < 0.05).

### Example 25

### LOX Pathway Blockers Enhance Photoaffinity Labeling of NTR1

NTR1 has been labeled using UV-activatable crosslinkers (Mazella et al., J. Biol. Chem., 263:144, 1988). Antiserum to NTR1 was used to immunoprecipitate NTR1 after UV-crosslinking of PC3 cells with ¹²⁵I-(4-azido-Phe⁶)-NT in the presence and absence of LOX pathway blockers using methods described in Example 4. Radioactivity associated with NTR1 was enhanced 2.6±0.3-fold and 2.7±0.3-fold by 50 μM NDGA and MK886, respectively. For each agent, the increase in immunoprecipitated radioactivity was similar to the increase in NT binding to PC3 cells at the dose used (FIG. 15B).

These results indicate that LOX pathway blockers enhanced NT binding by increasing the association of ¹²⁵I-NT with NTR1; however, they do not rule out possible interactions with other NT receptors.

### Example 26

### Cell-surface Binding Versus Internalization

Acid washing of PC3 cells after binding of ¹²⁵I-NT to the cells indicated that 72±2% of the ¹²⁵I-NT was internalized (n=8). The surface-bound ¹²⁵I-NT and the internalized ¹²⁵I-NT were both dose-dependently enhanced by NDGA (FIG. 16A) and MK886 (FIG. 16B). Internalization, as percentage of total binding, was unchanged by these agents (range for all groups: 68-73%). NDGA and MK886 also enhanced ¹²⁵I-NT binding similarly under conditions where internalization was fully inhibited by 10 μM phenylarsine oxide (results not shown). In addition, the rate of internalization at 37° C. for cell-surface ¹²⁵I-NT, previously bound to cells at 4° C., was unaffected by 50 μM NDGA [uptake rate (%/min): control, 9.5±0.8; NDGA, 10.2±0.9; n=8].

These results indicate that LOX pathway blockers increased cellular NT binding by enhancing the interaction of NT with NTR1. As was expected, these drugs also increased the total amount of NT-NTR complex internalized by the cell; however, they did not alter the rate of internalization or the percentage of bound NT that was internalized.

### Example 27

### NTR1 Affinity versus NTR1 Number

LOX pathway blockers enhanced binding and increased the steepness of the NT displacement curve. When NT displacement data were expressed as % maximal binding, methods in Example 3. For binding assays, PC3 cells were treated 10 minutes with agents, and NT receptor binding was evaluated at 37° C. using methods described in Examples 1 and 2.

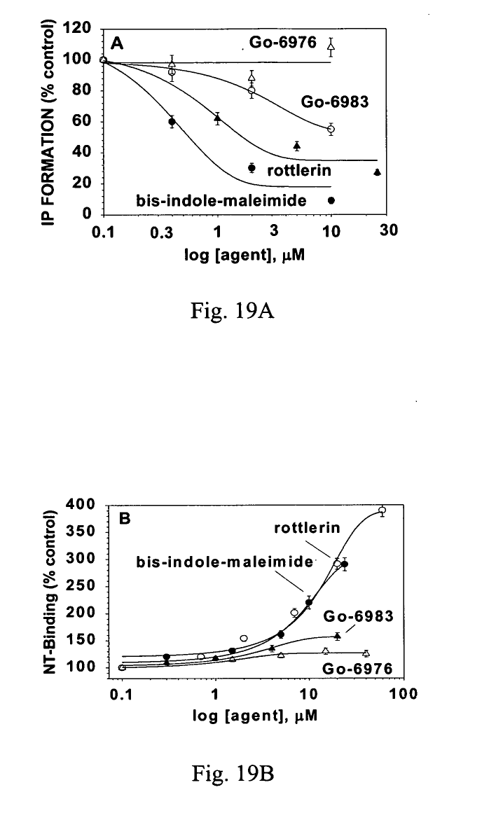

Inhibitors of novel PKCs inhibited NT-induced IP formation (FIG. 19A) and enhanced NT binding up to 4-fold. (FIG. 19B). The order of efficacy in the two assays correlated (BIS≅rottlerin>Go6983>>Go6976), indicating involvement of novel PKCs (δ,ε,η,θ) but not conventional PKCs (α, β1, β2, γ) in the effects.

The results demonstrate that inhibitors of novel PKCs mimic the effects of CCBs, DHPs, LOX inhibitors, polyphenols and other antioxidants.

### Example 29

### Inhibitors of Mitochondrial Function Enhance NT Binding

The mitochondrial electron transport chain is the most notable source of ROS in most cells (McLennan et al., J. Bioenerg. Biomemb., 32:153-162, 2000). To further examine the link between ROS formation and NTR function, we used inhibitors of mitochondrial function to reduce cellular ROS and tested the effects on NT binding and IP formation. The methods used were similar to those described in Example 28. Carbonyl cyanide 3-chlorophenylhydrozone (CCCP), antimycin A, myxothiazol, and rotenone were obtained from Sigma, dissolved at 10 mM in DMSO and diluted just before use.

Rotenone (inhibitor of complex I), antimycin A and myxothiazol (inhibitors of complex III), and CCCP (H⁺ ionophore) all of which would be expected to alter ROS production, elevated NT binding by as much as 3-fold. The EC50 for the increase in NT binding was: rotenone (38 μM), antimycin A (32 μM), myxothiazol (16 μM) and CCCP (23 μM). These results indicate that inhibitors of mitochondrial ROS production are similar to antioxidants in that they enhance cellular NT binding.

### Example 30

### Phenolic Agonists of Estrogen Receptors Enhance NT Binding

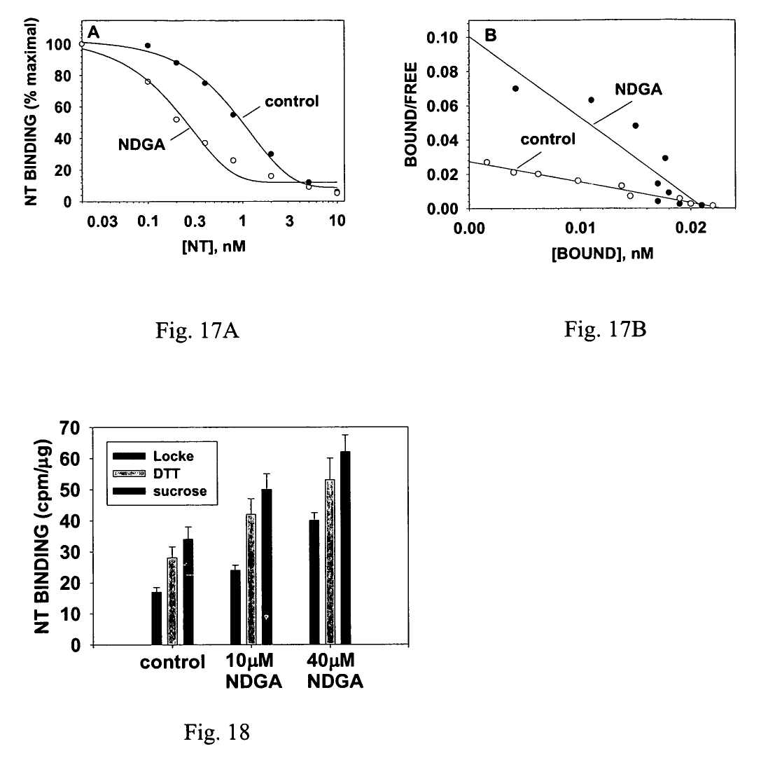

Estradiol and other estrogen receptor agonists and modulators possess phenolic hydroxyl group(s) that can produce antioxidative effects (Obach, Drug Metab. Disp., 32:89-97, 2004). Many of these compounds have important effects on the initiation and proliferation of various cancers (Kim et al., Cancer Res., 62:5365-5369, 2002). LOX pathway blockers shifted the displacement curves to the left by a factor of ≅3 (FIG. 18A). In three experiments, the average Ki for NT was decreased from 1.0±0.09 nm (control) to 0.28±0.05 nm (50 μM NDGA; p<0.01) and 0.35±0.05 nm (50 μM MK886; p<0.01). Scatchard analyses indicated that NDGA and MK886 increased the affinity of NTR1 for NT without changing receptor number (FIG. 17B; Table 5).

Taken together, these results indicated that LOX pathway blockers shifted NTR1 towards a state that displayed an increased affinity for the agonist NT.

TABLE 11
Effects of LOX Pathway Blockers on NT Binding Parameters

| | Bmax ^{b} | Ki for NT ^{b} |
|---|---|---|
| Agent ^{a} | (fmol/mg) | (nM) |
| control | 145 ± 9 | 1.0 ± 0.09 |
| NDGA | 128 ± 10 | 0.28 ± 0.03** |
| MK886 | 143 ± 12 | 0.35 ± 0.04** |

^{a} PCS cells were pretreated 10 minutes with 50 μM concentrations of each agent or vehicle control. ¹²⁵I-NT (10⁵ cpm, 50 pM) was added and specific binding was measured at 37° C.
^{b} Scatchard analyses were performed using 12 concentrations of NT and results for Bmax and Ki (mean ± SEM) were from at least three experiments for each agent.
**Result is significantly different from control (p < 0.01).

### Example 28

### PKC-Inhibitors Enhance NT Binding and Inhibit IP Formation

Protein kinase C is a family of enzymes that play key roles in tumor promotion and progression (Gopalakrishna, et al., Free Rad. Biol. Med., 28:1349-1361, 2000). Activation of PKC can stimulate cellular production of reactive oxygen species (ROS) (Inoguchi et al., J. Am. Soc. Nephrol., 14:S227-S232, 2003). Antioxidants can inactivate PKC, and this may be responsible for the inhibitory effects of antioxidants on cancer cell growth and tumor promotion (Gopalakrishna et al., J. Nutr., 132:3819S-3823S, 2002). These findings led us to investigate the effects of PKC inhibitors on NT binding and IP formation.

For IP assays, PC3 cells were treated 10 minutes with PKC inhibitors (from Sigma), then stimulated 30 minutes with NT before IP levels were evaluated using Therefore, it was of interest to assess their effects on NT binding. α-estradiol, β-estradiol and diethystilbestrol (DES), obtained from Sigma, were dissolved at 10 mM in DMSO just before use. PC3 cells were exposed to various concentrations (2 μM to 200 μM) of these agents (DMSO as control) for 10 minutes, and NT binding was measured as previously described. Each agent increased NT binding dose-responsively to a maximum of 305% (DES), 224% (β-estradiol), and 175% α-estradiol). The potency order was DES>β-estradiol>α-estradiol, with each agent increasing the binding by 75% at concentrations of 6 μM, 30 μM and 150 μM, respectively. The higher potency of DES can be attributed to the presence of the bisphenol structure, which resembles the structures of resveratrol and luteolin (antioxidants in Table 5). These results are in keeping with our other findings that indicate that NT binding is enhanced by compounds that produce antioxidative effects and furthermore, they suggest that the antioxidative property can be associated with anticancer activity.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A combination comprising:
(i) one or more labeled or conjugated neurotensin receptor ligands; and
(ii) one or more calcium channel blockers or one or more antioxidants, or both a calcium channel blocker and an antioxidant.

2. The combination of claim 1, wherein the one or more neurotensin receptor ligands are in a first container, and the one or more calcium channel blockers are in a second container.

3. The combination of claim 1, wherein the one or more neurotensin receptor ligands are in a first container, and the one or more antioxidants are in a second container.

4. The combination of claim 1, wherein the neurotensin receptor ligand is labeled with a moiety suitable for imaging.

5. The combination of claim 1, wherein the neurotensin receptor ligand is conjugated to an anti-tumor agent.

6. The combination of claim 1, wherein the calcium channel blocker is selected from the group consisting of felodipine, nicardipine, nitrendipine, nifedipine (NIF), nimodipine, phloretin, verapamil, SKF-96365, miconazole, trifluoperazine, chlorpromazine, and derivatives thereof.

7. The combination of claim 1, wherein the antioxidant is a member of the antioxidant class selected from the group consisting of: dihydropyridines (DHPs), polyphenolic antioxidants, flavonoids, retinoids, isoprenoids, glycolytic inhibitors, mitochondrial inhibitors, flavoprotein oxidase inhibitors, iron/zinc chelators, protein kinase-c inhibitors, tyrosine kinase inhibitors, inhibitors of glycogen synthase kinase, and estrogen agonists.

8. A method of screening for a tumor that expresses a higher level of neurotensin receptors than surrounding tissues in a subject, the method comprising:
administering to a subject (i) a labeled neurotensin receptor ligand and (ii) a calcium channel blocker or an antioxidant selected from the group consisting of dihydropyridines (DHPs), polyphenolic antioxidants, flavonoids, retinoids, isoprenoids, glycolytic inhibitors, mitochondrial inhibitors, flavoprotein oxidase inhibitors, iron/zinc chelators, protein kinase-c inhibitors, tyrosine kinase inhibitors, inhibitors of glycogen synthase kinase, and estrogen agonists, wherein the conjugated neurotensin receptor ligand is administered before, during, or after administration of the calcium channel blocker or antioxidant;
subsequently imaging at least a portion of the subject; and
screening for an increased concentration of labeled neurotensin receptor ligand, relative to tissues surrounding the increased concentration within the imaged portion of the subject;
wherein an increase in concentration of labeled neurotensin receptor ligand indicates that the subject has a tumor expressing a higher level of neurotensin receptors relative to the surrounding tissues.

9. The method of claim 8, wherein a calcium channel blocker is administered.

10. The method of claim 8, wherein an antioxidant is administered.

11. The method of claim 8, wherein the ligand is selected from the group consisting of neurotensin; a neurotensin fragment comprising neurotensin (8-13); neurotensin or an analog or fragment thereof with a substitution at one or more of the following positions: Arg 8, Arg 9, Pro 10, or Tyr 11, Ile 12, or Leu 13; a neurotensin with tryptophan substitution for Tyr 11; a neurotensin analog or fragment thereof; MP-2530; Neuromedin-N; xenopsin; xenin; histamine releasing peptide; SR48692; SR142948A; and levocabastine.

12. The method of claim 8, wherein the label is selected from the group consisting of a paramagnetic ion, a radioactive moiety, a fluorescent moiety, and a chromophore.

13. The method of claim 8, wherein the imaging comprises performing an imaging method selected from the group consisting of radioactive scanning, magnetic resonance imaging, or fluorescence imaging.

14. The method of claim 8, wherein the calcium channel blocker is selected from the group consisting of felodipine, nicardipine, nitrendipine, nifedipine (NIF), nimodipine, phloretin, verapamil, SKF-96365, miconazole, trifluoperazine, chlorpromazine, and derivatives thereof.

15. A method of treating a tumor, the method comprising administering to a subject diagnosed with a tumor: (i) an effective amount of a calcium channel blocker or an antioxidant or both and (ii) a therapeutic amount of a neurotensin receptor ligand conjugated to an anti-tumor agent, wherein the conjugated neurotensin receptor ligand is administered before, during, or after administration of the calcium channel blocker or antioxidant.

16. The method of claim 15, wherein a calcium channel blocker is administered.

17. The method of claim 15, wherein an antioxidant is administered.

18. The method of claim 15, wherein the tumor is selected from the group consisting of a Ewing's sarcoma, a myeloma, an astrocytoma, a lung tumor, a colon tumor, an ovarian tumor, a pancreatic tumor, a prostate tumor.

19. The method of claim 15, wherein the ligand is selected from the group consisting of neurotensin; a neurotensin fragment comprising neurotensin (8-13); neurotensin or an analog or fragment thereof with a substitution at one or more of the following positions: Arg 8, Arg 9, Pro 10, or Tyr 11, Ile 12, or Leu 13; a neurotensin with tryptophan substitution for Tyr 11; a neurotensin analog or fragment thereof; MP-2530; Neuromedin-N; xenopsin; xenin; histamine releasing peptide; SR48692; SR142948A; and levocabastine.

20. The method of claim 15, wherein the anti-tumor agent is selected from the group consisting of: a chemotherapeutic, a radiotherapeutic, a proapoptotic agent, a cytotoxic agent, or a cytostatic agent.

21. A method of identifying a cell expressing a neurotensin receptor, the method comprising:
(a) contacting a cell with a neurotensin receptor ligand;
(b) before, during, or after (a), contacting the cell with one or a calcium channel blocker or an antioxidant or both in an amount sufficient to enhance binding of the neurotensin receptor ligand to a neurotensin receptor; and
(c) monitoring enhanced binding of the neurotensin receptor ligand to the cell; wherein neurotensin receptor ligand binding to the cell indicates that the cell expresses a neurotensin receptor.

22. The method of claim 21, wherein the calcium channel blocker is selected from the group consisting of felodipine, nicardipine, nitrendipine, nifedipine (NIF), nimodipine, phloretin, verapamil, SKF-96365, miconazole, trifluoperazine, chlorpromazine, and derivatives thereof.

23. The method of claim 21, wherein the cell is selected from the group consisting of: Ewing's sarcoma cells, myeloma cells, astrocytoma cells, lung cancer cells, colon cancer cells, ovary cancer cells, pancreas cancer cells, and prostate cancer cells.

24. The method of claim 21, wherein the neurotensin receptor ligand is labeled with label selected from the group consisting of a radioactive moiety, a fluorescent moiety, a chromophore, a detectable enzyme, and an antigen.
